(19) United States
(12) Patent Application Publication
(10) Pub. No.: US 2013/0123508 A1
(43) Pub. Date: May 16, 2013

(54) NOVEL NONLINEAR CHROMOPHORES ESPECIALLY SUITED FOR USE IN ELECTRO-OPTICAL MODULATION

(71) Applicants: Yann Bretonniere, La Mulatiere (FR); Chantal Andraud, Genas (FR); Wissam Buron, Olivet (FR)

(72) Inventors: Yann Bretonniere, La Mulatiere (FR); Chantal Andraud, Genas (FR); Wissam Buron, Olivet (FR)

(73) Assignee: ECOLE NORMALE SUPERIEURE DE LYON, LYON CEDEX 07 (FR)

(21) Appl. No.: 13/521,777

(22) PCT Filed: Jan. 18, 2011
(86) PCT No.: PCT/FR2011/050087

§ 371 Date: Oct. 10, 2012

(30) Foreign Application Priority Data
Jan. 22, 2010 (FR) 1050436

(51) Int. Cl. C07D 405/08; C07D 405/14; C07F 7/10

(52) U.S. Cl. CPC C07D 405/08; C07F 7/10; C07D 405/14
USPC 548/255; 548/454; 548/406

(57) ABSTRACT

The present invention relates to heptamethine hemicyanine chromophores of formula (I): where R₁, R₂, R₃, R₄, R′₄, R₅, R′₅, R₆, R′₆, R₇, X, Y₁ and Y₂ are as defined in claim 1, to the method for preparing same as well as to the use of such chromophores for the second-order nonlinear optical property thereof.

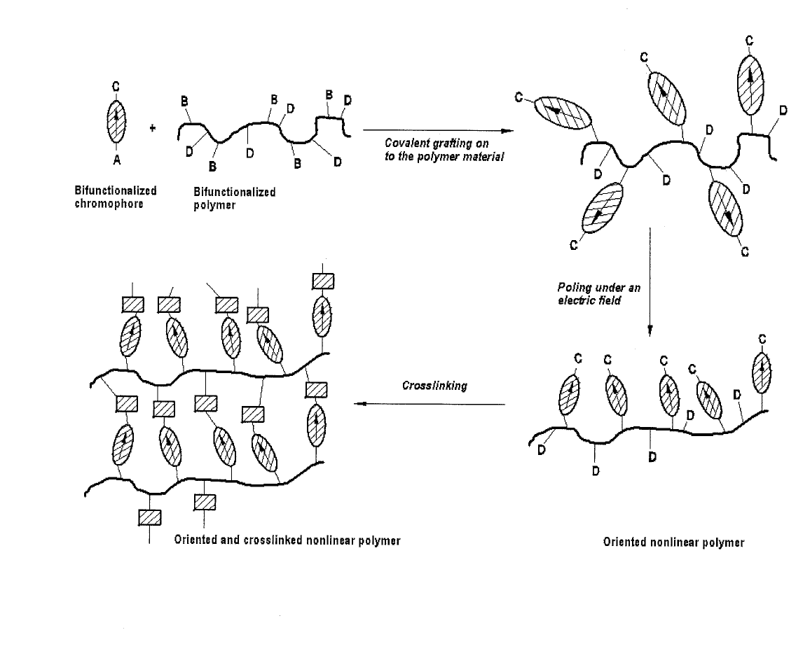

The present invention relates to novel nonlinear chromophores that are particularly suitable for applications in electro-optical modulation.

In the field of electro-optics, organic nonlinear chromophores having a very high quadratic hyperpolarizability β are sought which can be chemically modified so as to be able to be incorporated into a polymer material capable of being crosslinked by said chromophores. Such molecules are indeed highly sought in order to constitute the active core of an electro-optical modulator. An electro-optical modulator is a key component of telecommunication and information systems, which modulates the light which it receives using the variation in the refractive index which certain nonlinear materials exhibit depending on the electric field to which they are subjected. The optical phenomenon used is a second-order nonlinear phenomenon which is therefore only possible in a non-centrosymmetric medium.

The nonlinear materials conventionally used in the current techniques for manufacturing electro-optical modulators are non-centrosymmetric inorganic crystals, the material most frequently used being lithium niobate (LiNbO₃). Over the past few years, a lot of effort has been made in order to improve the performance of these modulators, mainly in terms of speed of transmission and passband, but the limitations intrinsic to the material were reached (high refractive index, low integrability with the integrated optical components and the optical fibers, expensive process for the manufacture of monocrystals and the like). Currently, the best lithium niobate-based modulators operate at 40 Gb/s with a passband of 30-35 GHz, but these values are no longer sufficient and this technology is difficult to integrate into the optical fiber technology.

The alternative to inorganic crystals is the use of modulators built with organic polymer materials in which the active nonlinear core is an organic molecule (chromophore) having a high second-order nonlinear activity (product μ.β, in which μ is the dipole moment of the molecule and β the vector part of the quadratic hyperpolarizability). Organic polymer materials have considerable advantages: (i) vast possibilities for engineering the nonlinear organic molecules, (ii) good integrability of the organic compounds with existing optical fibers, (iii) potential to increase the passband up to 100 GHz, (iv) low cost of use and of forming. Accordingly, the search for novel organic chromophores having excellent microscopic quadratic nonlinear properties (high μ.β coefficient) has developed considerably over the past twenty-five years, both academically and industrially.

The most advantageous molecules for this purpose are non-centrosymmetric dipolar nonlinear chromophores, that is to say in which an electron-donating group and an electron-attracting group are linked by a π-conjugated system, so as to obtain non-negligible values of μβ. The nonlinear material used is obtained by introducing the active molecule into an organic polymer, either as a host-guest system (without covalent attachment), or by covalently grafting the molecule on to the polymer, before or after polymerization, it being necessary to maintain the non-centrosymmetry of the material on a macroscopic scale so as to observe the electro-optical nonlinear effect.

The main problem is the transposition of the excellent molecular nonlinear properties on the scale of the material (macroscopic coefficient χ²). The non-centrosymmetric orientation in the material is obtained by the electric poling technique which consists in orienting all the dipoles by applying an electric field at a temperature greater than the glass transition temperature of the polymer used. The field is cut when the polymer is again rigid after the temperature has been reduced. Materials with very good properties may thus be obtained, but they are far from competing with the existing inorganic materials because the orientation rapidly disappears because of strong electrostatic interactions between the permanent dipoles of the chromophores, which tend to align them head-to-tail, and therefore to bring about a return to a centrosymmetric arrangement of the molecules. It has been shown that the grafted systems are a lot more stable than the simple host-guest systems. Furthermore, better stability of orientation is also observed when the polymer is rigidified by crosslinking after poling. This crosslinking may be performed either on the polymer without involving the chromophore, or by the chromophore. This requires the functionalization of the nonlinear chromophore by at least two orthogonal chemical functional groups. Thus, for an organic chromophore to be capable of being used as active core in a crosslinkable polymer material, it should have, in addition to ease of synthesis for industrial use on a scale of several grams, good second-order optical properties (characterized by the quadratic hyperpolarizability β or the product μβ, the scalar product of the dipole moment μ and of the vector component of the quadratic hyperpolarizability β) and it should be thermally and chemically stable. Furthermore, in order to be able to keep the orientation of the molecules after poling, it is preferable that the molecule is capable of being covalently grafted on to the polymer material. It should therefore have a suitable chemical functional group. A second chemical functional group, orthogonal to the preceding one and serving for the crosslinking of the polymer after orientation of the chromophore, is also advantageous.

The most efficient organic chromophores described to date, for this type of application, are CLD-1 and its analog FTC of formula:

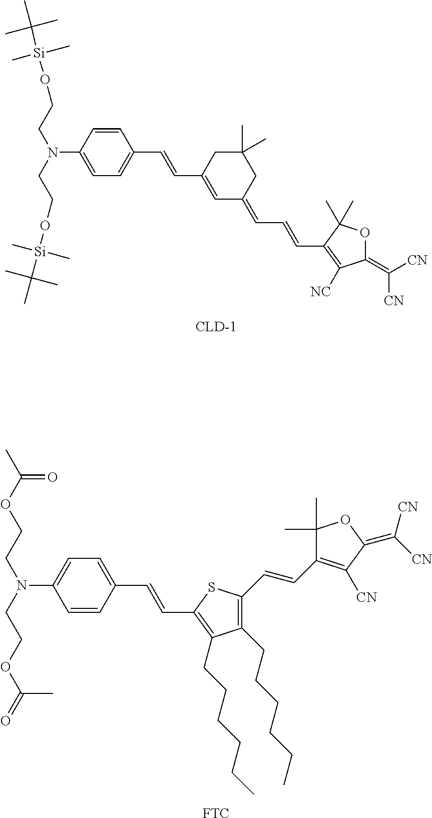 [SMILES: CC1(C)CC(/C=C/c2ccc(N(CCO[Si](C)(C)C(C)(C)C)CCO[Si](C)(C)C(C)(C)C)cc2)=CC(=C/C=C/C2=C(C#N)C(=C(C#N)C#N)OC2(C)C)/C1 ; CCCCCCc1c(/C=C/C2=C(C#N)C(=C(C#N)C#N)OC2(C)C)sc(/C=C/c2ccc(N(CCOC(C)=O)CCOC(C)=O)cc2)c1CCCCCC ; "FTC"]

which constitute the active core of an electro-optical modulator prototype (Shi, Y. et al. Science 2000, 288, 119-122 and Zhang, C. et al. Chem. Mater. 2001, 13, 3043-3050, Ermer et al. Adv. Funct. Mater. 2002, 12 (9), 605-610). CLD-1 has remarkable thermal and photophysical stability, as well as remarkable second-order nonlinear optical properties (μ.β=14 065×10⁻⁴⁸ esu, measured at 1.907 μm in solution in THF according to Zhang, C. et al. supra which is among the highest described), but can only be used in a host-guest system. Another molecule, a dipolar porphyrin of zinc (LeCours, S. M. et al. J. Am. Chem. Soc. 1996, 118, 1497-1503) as represented below:

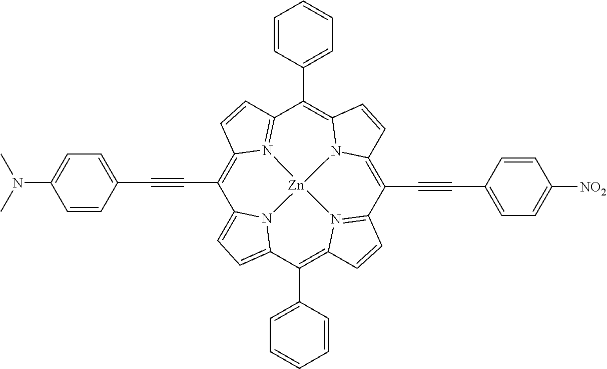

has even more remarkable quadratic nonlinear optical properties (μ.β=55 000×10⁻⁴⁸ esu, at 1.907 μm), but no functional active system built on this unit has so far been described.

Many studies by several American teams, especially the one by Alex Jen at the University of Washington in Seattle, have made it possible to improve the structure of CLD-1 by introducing various units into the accepting group, by substituting the central ring at the 2-position and introducing functional groups on to the oxygen atoms of the electron-donating part. Functional compounds have been described which allow covalent incorporation into various polymer matrices by postfunctionalization of the polymer on polyimides (Lindsay et al., Polymer 2007, 48, 6605-6616), polystyrenes (Luo et al. Advanced Materials 2003, 15, 1635-1638), polyurethanes (Briers et al, Polymer, 2004, 45, 19-24 and Zhang et al Macromolecules (Washington D.C. US), 2001, 34, 235-243), or by direct polymerization on polyperfluorocyclobutanes (Budy et al., Journal of Physical Chemistry C, 2008, 112, 8099-8104). These studies have also made it possible to develop polyfunctional compounds containing functional dendrimers which serve for the isolation of a site and for the crosslinking (Tian et al. Macromolecules (Washington D.C. US), 2007, 40, 97-104, Luo et al. Macromolecules (Washington D.C. US), 2004, 37, 248-250 and Zhou et al. Advanced Materials 2009, 21, 1976-1981), as well as functional groups, such as anthracene, which serve either for incorporation into the polymer (Zhou et al, supra and Shi et al. Macromolecules (Washington D.C. US), 2009, 42, 2438-2445), or for the crosslinking of the polymer (Kim et al. Advanced Materials 2006, 18, 3038-3042) by controlled Diels-Alder reaction. By virtue of the covalent grafting and the rigidification of the systems, the values of coefficients r₃₃ may be up to 250 mp/V.13.

However, these molecules pose a major problem: their synthesis is long and difficult. The synthesis of CLD-1 is described by Zhang, C. et al. in Chem. Mater. 2001, 13, 3043-3050, and in U.S. Pat. No. 6,348,992, and by Dalton et al. in U.S. Pat. No. 6,361,717. Scheme 1 represents the synthesis of CLD-1 as described by Zhang, C. et al. in Chem. Mater. 2001, 13, 3043-3050.

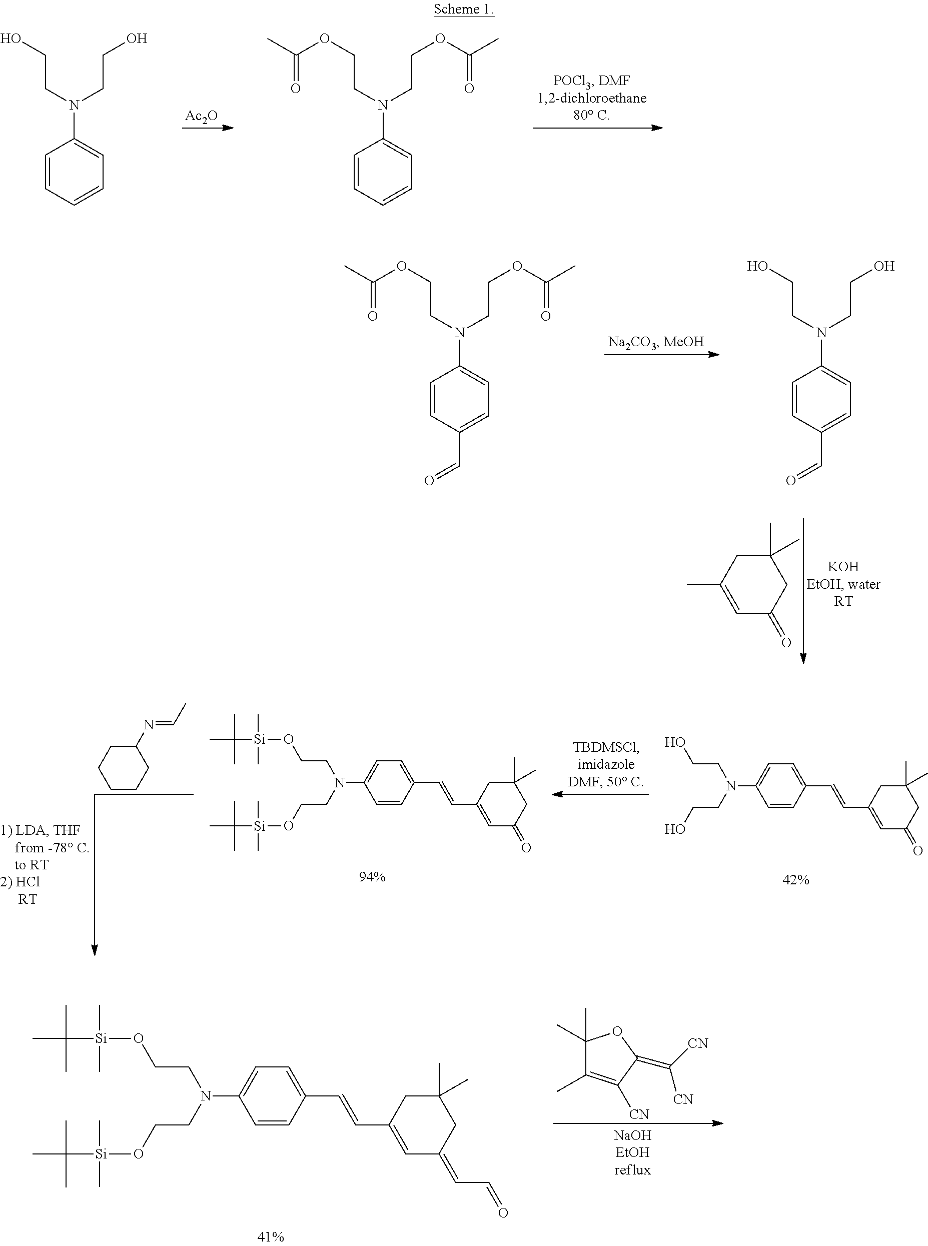

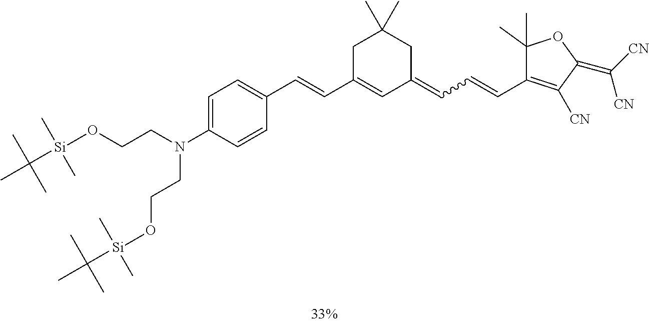 [SMILES: CC1(C)CC(=CC=CC2=C(C#N)C(=C(C#N)C#N)OC2(C)C)C=C(/C=C/c2ccc(N(CCO[Si](C)(C)C(C)(C)C)CCO[Si](C)(C)C(C)(C)C)cc2)C1]

This synthesis comprises eight steps and corresponds to an overall yield of only 6%. The length and difficulties of synthesis are even greater for the polyfunctional compounds. The key step is the extension of a dienone unit into a trienal. This aspect, which is linked to the synthesis of this series of compounds, makes the production of large quantities of products, necessary for industrial application, problematic.

Patent application WO 2004/111043 describes compounds presented as having second-order nonlinear optical properties. It should nevertheless be underlined that the compounds presented on page 31 of D1 containing a benzothiazolidinylidene group do not exhibit suitable properties in terms of stability and solubility in order to be incorporated into a polymer. Now, these properties are essential in the context of the invention, which proposes to provide novel organic chromophones exhibiting good second-order nonlinear optical properties and which can be incorporated into polymer materials. Moreover, no result reflecting the second-order nonlinear optical properties is given for the compounds described in WO 2004/111043, given that it is not possible to measure them. Indeed, the compounds exemplified in WO 2004/111043 are insoluble in virtually all solvents and no measurements are possible: that is why only theoretical values are given.

In this context, the present invention proposes to provide novel nonlinear organic chromophores which can be obtained according to a relatively simple method of synthesis, in particular compared with the one used for CLD-1 and its derivatives.

The chromophores according to the invention should also exhibit good second-order nonlinear optical properties.

Another object of the invention is to provide chromophores exhibiting second-order nonlinear optical properties, whose stability and solubility are compatible with their incorporation into polymer materials.

Another object of the invention is to provide chromophores which also exhibit second-order nonlinear optical properties when they are incorporated into a polymer material.

The subject of the invention is therefore heptamethine hemicyanine type chromophores of formula (I):

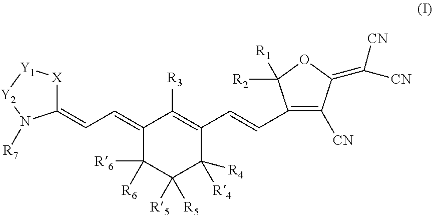

in which:
R₁ and R₂, which are identical or different, represent, each independently of each other, an alkyl group of 1 to 12 carbon atoms, a cycloalkyl group of 3 to 7 carbon atoms, a perfluoroalkyl group of 1 to 12 carbon atoms or a phenyl group, it being possible for said alkyl, perfluoroalkyl, cycloalkyl and phenyl groups to be unsubstituted or substituted with one or more substituents chosen from hydroxyl —OH, carboxylic acid —COOH optionally in protected form, amine —NH₂ optionally in protected form, azide —N₃ and thiol —SH groups,
R₃ represents a hydrogen, chlorine, fluorine, bromine or iodine atom or a group chosen from the groups:
   alkyl of 1 to 12 carbon atoms, cycloalkyl of 3 to 7 carbon atoms or perfluoroalkyl of 1 to 12 carbon atoms, it being possible for said alkyl, perfluoroalkyl and cycloalkyl groups to be unsubstituted or substituted with one or more substituents chosen from chlorine, bromine and iodine atoms, and the azide —N₃, hydroxyl —OH, thiol —SH, carboxylic acid —COOH optionally in protected form and —NH₂ optionally in protected form, groups,
   —C≡CR₈, in which R₈ represents a hydrogen atom or a trialkylsilyl group or a group chosen from alkyl groups of 1 to 12 carbon atoms, cycloalkyl groups of 3 to 7 carbon atoms or perfluoroalkyl groups of 1 to 12 carbon atoms, it being possible for said alkyl, perfluoroalkyl and cycloalkyl groups to be unsubstituted or substituted with one or more substituents chosen from chlorine, bromine and iodine atoms, and the azide —N₃, hydroxyl —OH, thiol —SH, carboxylic acid —COOH optionally in protected form, and —NH₂ optionally in protected form, groups,
   (1H-1,2,3-triazol-4-yl) of formula:

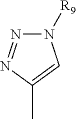

in which R₉ represents a group chosen from alkyl groups of 1 to 12 carbon atoms, cycloalkyl groups of 3 to 7 carbon atoms, perfluoroalkyl groups of 1 to 12 carbon atoms and phenyl groups, it being possible for said alkyl, perfluoroalkyl, cycloalkyl and phenyl groups to be unsubstituted or substituted with one or more substituents chosen from chlorine, bromine and iodine atoms, and the azide —N₃, hydroxyl —OH, thiol —SH, carboxylic acid —COOH optionally in protected form and —NH₂ optionally in protected form, groups, and
—O-phenyl or —S-phenyl, optionally substituted with a chlorine, bromine, iodine or fluorine atom, or with a group chosen from the groups:
   azide —N₃,
   hydroxyl —OH,
   thiol —SH,
   carboxylic acid —COOH optionally in protected form,
   —NH₂ optionally in protected form,
   alkyl of 1 to 6 carbon atoms,
   —(CH₂)ₙ—OH, —(CH₂)ₙ—SH, —(CH₂)ₙ—N₃, —(CH₂)ₙ—NH₂ optionally in protected form, —(CH₂)ₙ—COOH optionally in protected form, where n may be equal to 1, 2, 3, 4, 5 or 6,
   —C≡CR₁₀, where R₁₀ represents a hydrogen atom or a trialkylsilyl group,
   (1H-1,2,3-triazol-4-yl) of formula:

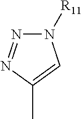

with R₁₁ which represents an alkyl group of 1 to 12 carbon atoms, a cycloalkyl group of 3 to 7 carbon atoms, a perfluoroalkyl group of 1 to 12 carbon atoms or a phenyl group, it being possible for said alkyl, perfluoroalkyl, cycloalkyl and phenyl groups to be unsubstituted or substituted with one or more substituents chosen from the hydroxyl —OH, carboxylic acid —COOH optionally in protected form, amine —NH₂ optionally in protected form, azide —N₃ and thiol —SH groups,
of the dendritic type of general formula:

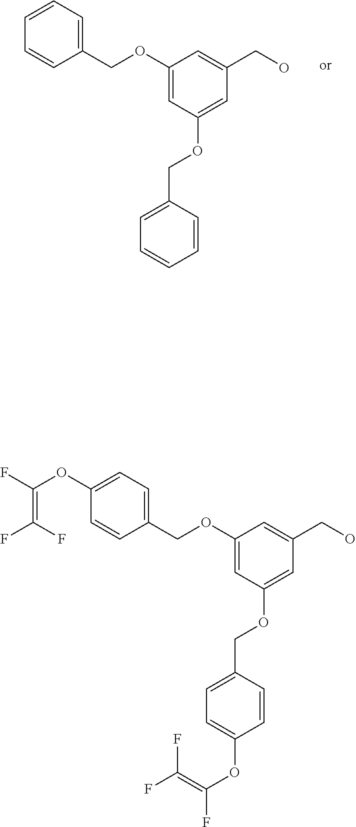

and
—C(O)NHR₁₂, with R₁₂ which represents a —(CH₂)ₚ—OH group with p which may be equal to 1, 2, 3, 4, 5 or 6, or a group chosen from alkyl groups of 1 to 12 carbon atoms, cycloalkyl groups of 3 to 7 carbon atoms, perfluoroalkyl groups of 1 to 12 carbon atoms and phenyl groups, it being possible for said alkyl, cycloalkyl, perfluoroalkyl and phenyl groups to be unsubstituted or substituted with one or more substituents chosen from hydroxyl —OH, carboxylic acid —COOH optionally in protected form, amine —NH₂ optionally in protected form,
—O—R₁₃ or —SR₁₃, with R₁₃ which represents an alkyl group of 1 to 12 carbon atoms or a cycloalkyl group of 3 to 7 carbon atoms, it being possible for said alkyl and cycloalkyl groups to be unsubstituted or substituted with one or more substituents chosen from hydroxyl —OH, carboxylic acid —COOH optionally in protected form, amine —NH₂ optionally in protected form, azide —N₃ and thiol —SH groups,
R₄, R₄, R₅, R′₅, R₆ and R′₆, which are identical or different, represent, each independently of each other:
   a hydrogen atom,
   a cyano group,
   a hydroxyl functional group —OH,
   a carboxylic acid functional group —COOH optionally in protected form,
   an alkyl group of 1 to 15 carbon atoms, a perfluoroalkyl group of 1 to 15 carbon atoms or a cycloalkyl group of 3 to 7 carbon atoms, it being possible for said alkyl, perfluoroalkyl and cycloalkyl groups to be unsubstituted or substituted with one or more substituents chosen from hydroxyl —OH, carboxylic acid —COOH optionally in protected form, and amine —NH₂ optionally in protected form, groups, or
   a phenyl group, optionally substituted with one or more substituents chosen from chlorine atoms and nitrile —CN, carboxylic acid —COOH optionally in protected form, hydroxyl —OH and amine —NH₂ optionally in protected form, groups,
or else R₅ and R′₅ are linked to each other in order to form the linkage —OCH₂CH₂O—; or R₅ and R′₅ form with the carbon atom with which they are linked a —C(O)— functional group, R₄, R′₄, R₆ and R′₆ being as defined above,
or else R₄ and R₆ are linked to each other in order to form an alkylene chain comprising 5 or 6 carbon atoms, R′₄, R₅, R′₅ and R′₆ being as defined above, it being understood that in this case, R₅ and R′₅ are not linked to each other in order to form the linkage —OCH₂CH₂O—,
X represents CRR′, O, S or Se, with R and R′, which are identical or different, which represent, each independently of each other, an alkyl group of 1 to 6 carbon atoms or a cycloalkyl group of 3 to 7 carbon atoms,
Y₁ and Y₂ form a linkage chosen from:

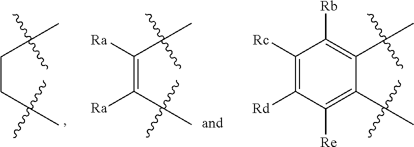

with Ra, which represents a hydrogen atom or a methyl group, and Rb, Rc, Rd and Re, which are identical or different, which represent, each independently of each other, a hydrogen atom, a hydroxyl group —OH, a carboxylic acid group —COOH optionally in protected form or an amine group —NH₂ optionally in protected form,
R₇ represents a group chosen from the groups:
   alkyl of 1 to 12 carbon atoms, cycloalkyl of 3 to 7 carbon atoms and perfluoroalkyl of 1 to 12 carbon atoms, it being possible for said alkyl, perfluoroalkyl and cycloalkyl groups to be unsubstituted or substituted with one or more substituents chosen from chlorine, bromine and iodine atoms, and the azide —N₃, hydroxyl —OH, thiol —SH, carboxylic acid —COOH optionally in protected form and —NH₂ optionally in protected form, groups,
   benzyl which is unsubstituted or substituted with one or more substituents chosen from chlorine, bromine and iodine atoms, and the groups:
      azide —N₃,
      hydroxyl —OH,
      carboxylic acid —COOH optionally in protected form,
      amine —NH₂ optionally in protected form,
      —C≡CR₁₃, with R₁₃, which represents a hydrogen atom, a trialkylsilyl group, an alkyl group of 1 to 12 carbon atoms, a cycloalkyl group of 3 to 7 carbon atoms or a perfluoroalkyl group of 1 to 12 carbon atoms, it being possible for said alkyl, perfluoroalkyl and cycloalkyl groups to be unsubstituted or substituted with one or more substituents chosen from hydroxyl —OH, carboxylic acid —COOH optionally in protected form, amine —NH₂ optionally in protected form, azide —N₃ and thiol —SH, groups,
      (1H-1,2,3-triazol-4-yl) of formula:

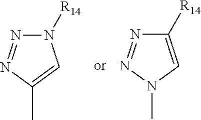

with R₁₄ which represents a group chosen from alkyl groups of 1 to 12 carbon atoms, cycloalkyl groups of 3 to 7 carbon atoms, perfluoroalkyl groups of 1 to 12 carbon atoms and phenyl groups, it being possible for said alkyl, cycloalkyl, perfluoroalkyl and phenyl groups to be unsubstituted or substituted with one or more substituents chosen from hydroxyl —OH, carboxylic acid —COOH optionally in protected form, amine —NH₂ optionally in protected form, azide —N₃ and thiol —SH, groups,
—(CH₂)ₘ—C(O)—R₁₅ with m which is equal to 1, 2, 3, 4, 5 or 6 and R₁₅ which represents —OH or a group —NHR₁₆ with R₁₆ which is chosen from the groups:
   phenyl optionally substituted with one or more substituents chosen from chlorine, bromine and iodine atoms, and azide groups —N₃, hydroxyl groups —OH, carboxylic acid groups —COOH optionally in protected form, —NH₂ groups optionally in protected form, and —C≡CR₁₇ groups, with R₁₇ which represents a hydrogen atom, a trialkylsilyl group, an alkyl group of 1 to 12 carbon atoms, a cycloalkyl group of 3 to 7 carbon atoms or a perfluoroalkyl group of 1 to 12 carbon atoms, it being possible for said alkyl, cycloalkyl and perfluoroalkyl groups to be unsubstituted or substituted with one or more substituents chosen from hydroxyl —OH, carboxylic acid —COOH optionally in protected form, amine —NH₂ optionally in protected form, azide —N₃ and thiol —SH, groups,
   (1H-1,2,3-triazol-4-yl) of formula:

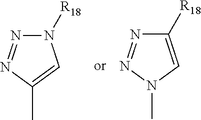

with R₁₈ which represents an alkyl group of 1 to 12 carbon atoms, a cycloalkyl group of 3 to 7 carbon atoms, a perfluoroalkyl group of 1 to 12 carbon atoms, or a phenyl group, it being possible for said alkyl, cycloalkyl, perfluoroalkyl and phenyl groups to be unsubstituted or substituted with one or more substituents chosen from hydroxyl —OH, carboxylic acid —COOH optionally in protected form, amine —NH₂ optionally in protected form, azide —N₃ and thiol —SH, groups,
—(CH₂)_{q}—OH or —(CH₂)_{q}—NH₂, optionally in protected form, with q which may be equal to 1, 2, 3, 4, 5 or 6,
   which contain a functional group allowing covalent bonding of the chromophore with a polymer, and/or a functional group allowing, once the chromophore is incorporated into a polymer, crosslinking of the latter, said polymer being in particular chosen from polymers of the polymethacrylate, polyimide, polystyrene, perfluorocyclobutane and polycarbonate type.

The compounds of formula (1a) and (1b):

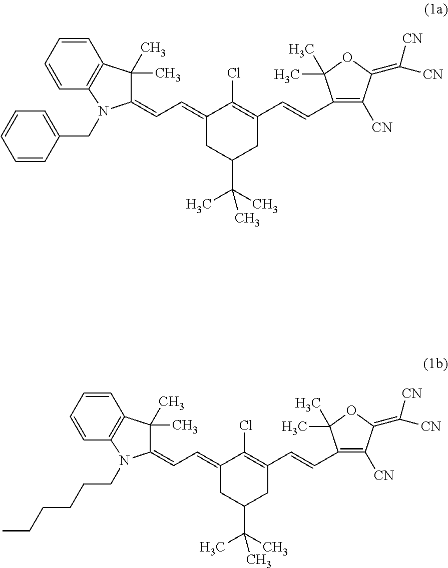 [SMILES: CC1(C)OC(=C(C#N)C#N)C(C#N)=C1/C=C/C1=C(Cl)C(=C/C=C2/N(Cc3ccccc3)c3ccccc3C2(C)C)/CC(C(C)(C)C)C1 ; CCCCCCN1/C(=C/C=C2\CC(C(C)(C)C)CC(/C=C/C3=C(C#N)C(=C(C#N)C#N)OC3(C)C)=C2Cl)C(C)(C)c2ccccc21]

do not belong to the family of compounds claimed: these compounds do not correspond to the definition of the compounds claimed in the context of the present invention, given that they do not contain any functional group allowing covalent bonding of the chromophore with a polymer, or a functional group allowing, once the chromophore is incorporated into a polymer, crosslinking of the latter.

The compounds (1a) and (1b) were developed for the optical limitation in the near infrared (Bouit et al. Chem. Mater. 2007, 19 (22), 5325-5335) and are described in the literature for their 3^{rd} order nonlinear optical properties: in the present case, the two-photon absorption properties which are of average efficacy in the case of these two molecules. From these results, it was not at all predictable that these molecules have second-order nonlinear optical properties with exceptional efficacy in solution in this domain, let alone that these second-order nonlinear properties would be preserved, when these chromophores are integrated within a polymer. In the context of the invention, the inventors have demonstrated that the compounds of formula (I) according to the invention exhibited 2^{nd} order nonlinear optical properties characterized by a high value of the scalar product μ.β where μ represents the dipole moment of the molecule and β the vector value of the quadratic hyperpolarizability, and could therefore, as a result, be used to manufacture an electro-optical modulator. In particular, it has been demonstrated that compound (1a) as doping at 20% by mass in polymethyl methacrylate (PMMA) gives, after orientation by corona poling, a nonlinear material with an r₃₃ value of 80 mp/V at 970 nm and good temporal stability. An r₃₃ value of 70 mp/V at 1550 nm is obtained with the same compound as doping at 20% by mass in PMMA-co-DR1. Consequently, the second-order nonlinear optical properties of the chromophores of formula (I) are transposed when the latter are incorporated into a polymer in order to form a material.

Furthermore, the compounds according to the invention, by virtue of their heptamethine hemicyanine structure, are obtained according to a chemistry that is quite different from that of CLD-1 and its derivatives. For equivalent functionalizations, their synthesis is much easier, comprises fewer steps and is therefore less expensive and more easily transposable on an industrial scale.

Accordingly, the subject of the present invention is also the use of a chromophore of formula (I) for its second-order nonlinear optical properties, and in particular for the manufacture of an electro-optical modulator. In this context, the chromophore will be incorporated into a polymer in order to manufacture an electro-optical modulator.

According to a variant embodiment, the compounds according to the invention correspond to the formula (Ia):

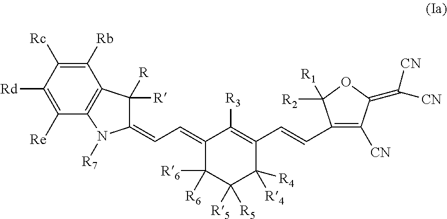

with R₁, R₂, R₃, R₄, R′₄, R₅, R′₅, R₆, R′₆, R₇, R, R′, Rb, Rc, Rd and Re as defined above.

According to particular variant embodiments, the chromophores of formula (I) and (Ia) have any of the following characteristics or a combination of these characteristics or all of the characteristics below, when they are not mutually exclusive:
R₁═R₂═CH₃,
R═R′═CH₃,
Rb=Rc=Rd=Re═H,
R₄═R′₄═R₆═R′₆═H, or R₄═R′₄═R₆═R′₆═—CH₃,
R₅ and R′₅ are defined as follows:
   R₅═R′₅═H; or
   R₅═R′₅═—CH₃; or
   R₅═H and R′₅ represents a methyl, ethyl, propyl, trifluoromethyl —CF₃, phenyl or tert-butyl group; or
   R₅ represents a cyano —CN group and R′₅ represents a phenyl group; or
   R₅═H and R′₅ represents a carboxyl —COOH group optionally in protected form; or
   R₅ and R′₅ are bonded to each other in order to form the linkage —OCH₂CH₂O—; or
   R₅ and R′₅ form with the carbon atom with which they are linked a functional group —C(O)—,
R₃ and/or R₇ comprise(s) a functional group chosen from the functional groups carboxylic acid —COOH optionally in protected form, hydroxyl —OH, azide —N₃, amine —NH₂ optionally in protected form, sulfhydryl (—SH), trifluorovinyloxy, ethenyl and the groups —C≡CRf, where Rf is a hydrogen atom or a trialkylsilyl group, and in particular:
   R₃ represents a chlorine, fluorine, bromine or iodine atom or a group chosen from the groups:
(1H-1,2,3-triazol-4-yl) of formula:

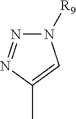

in which R₉ represents a group chosen from alkyl groups of 1 to 12 carbon atoms, and
—O-phenyl or —S-phenyl, optionally substituted with a chlorine, bromine, iodine or fluorine atom, or with a group chosen from the groups:
   —(CH₂)ₙ—OH, —(CH₂)ₙ—N₃, where n may be equal to 1, 2, 3,
   —C≡CR₁₀, where R₁₀ represents a trialkylsilyl group, and in particular trimethylsilyl, and
R₇ represents a chlorine, fluorine, bromine or iodine atom or a group chosen from the groups:
R₇ represents a group chosen from the groups:
   benzyl which is unsubstituted or substituted with one or more substituents chosen from chlorine, bromine and iodine atoms, and the groups —C≡CR₁₃, with R₁₃, which represents a trialkylsilyl group, and in particular trimethylsilyl,
   —(CH₂)ₘ—C(O)—R₁₅, with m which is equal to 3, 4, 5 or 6 and R₁₅ which represents —OH or a group —NHR₁₆, with R₁₆ which is chosen from phenyl groups substituted with one or more substituents chosen from chlorine, bromine and iodine atoms, and azide groups —N₃, trialkylsilyl groups, alkyl groups of 1 to 12 carbon atoms substituted with one or more substituents chosen from hydroxyl —OH and azide —N₃ groups, it being understood that at least one of the groups R₃ and R₇ comprises a functional group chosen from the functional groups carboxylic acid —COOH, hydroxyl —OH, azide —N₃, amine —NH₂ in a protected form, and the groups —C≡CRf, where Rf is a trialkylsilyl group.

The following compounds are examples of compounds of formula (Ia) and form an integral part of the invention:
2-(4-((E)-2-((E)-3-((E)-2-(1-benzyl-3,3-dimethylindolin-2-ylidene)ethylidene)-5-tert-butyl-2-(phenoxy)cyclohex-1-enyl)vinyl)-3-cyano-5,5-dimethylfuran-2(5H)-ylidene)malononitrile, compound (2), of formula:

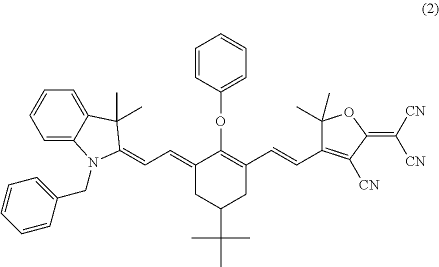 [SMILES: CC1(C)OC(=C(C#N)C#N)C(C#N)=C1/C=C/C1=C(Oc2ccccc2)C(=C/C=C2/N(Cc3ccccc3)c3ccccc3C2(C)C)/CC(C(C)(C)C)C1]

2-(4-((E)-2-((E)-3-((E)-2-(1-benzyl-3,3-dimethylindolin-2-ylidene)ethylidene)-5-tert-butyl-2-(4-(hydroxymethyl)phenoxy)cyclohex-1-enyl)vinyl)-3-cyano-5,5-dimethylfuran-2(5H)-ylidene)malononitrile, compound (3), of formula:

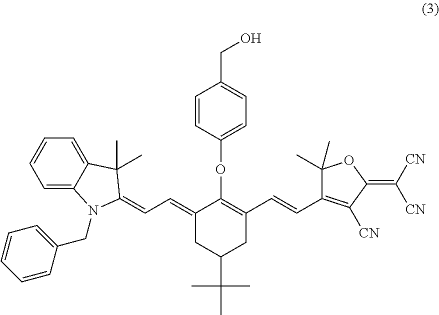 [SMILES: CC1(C)OC(=C(C#N)C#N)C(C#N)=C1/C=C/C1=C(Oc2ccc(CO)cc2)C(=C/C=C2/N(Cc3ccccc3)c3ccccc3C2(C)C)/CC(C(C)(C)C)C1]

2-(4-((E)-2-((E)-3-((E)-2-(1-benzyl-3,3-dimethylindolin-2-ylidene)ethylidene)-5-tert-butyl-2-((trimethylsilyl)ethynyl)cyclohex-1-enyl)vinyl)-3-cyano-5,5-dimethylfuran-2(5H)-ylidene)malononitrile, compound (4), of formula:

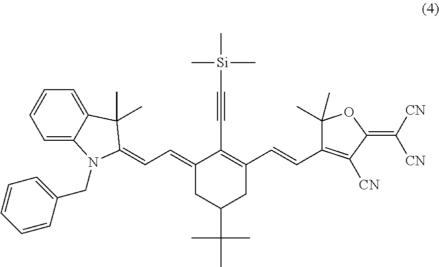 [SMILES: CC1(C)OC(=C(C#N)C#N)C(C#N)=C1/C=C/C1=C(C#C[Si](C)(C)C)C(=C/C=C2/N(Cc3ccccc3)c3ccccc3C2(C)C)/CC(C(C)(C)C)C1]

2-(4-((E)-2-((E)-3-((E)-2-(1-benzyl-3,3-dimethylindolin-2-ylidene)ethylidene)-5-tert-butyl-2-(1-hexyl-1H-1,2,3-triazol-4-yl)cyclohex-1-enyl)vinyl)-3-cyano-5,5-dimethylfuran-2(5M-ylidene)malononitrile, compound (5), of formula:

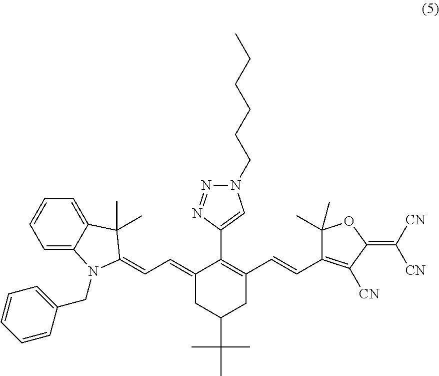 [SMILES: CCCCCCn1cc(C2=C(/C=C/C3=C(C#N)C(=C(C#N)C#N)OC3(C)C)CC(C(C)(C)C)C/C2=C\C=C2\N(Cc3ccccc3)c3ccccc3C2(C)C)nn1]

2-(4-((E)-2-((E)-3-((E)-2-(1-benzyl-3,3-dimethylindolin-2-ylidene)ethylidene)-2-(4-bromophenylthio)-5-tert-butylcyclohex-1-enyl)vinyl)-3-cyano-5,5-dimethylfuran-2(5M-ylidene)malononitrile, compound (6), of formula:

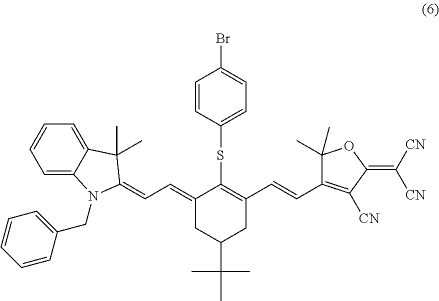 [SMILES: CC1(C)OC(=C(C#N)C#N)C(C#N)=C1/C=C/C1=C(Sc2ccc(Br)cc2)C(=C/C=C2/N(Cc3ccccc3)c3ccccc3C2(C)C)/CC(C(C)(C)C)C1]

6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)hexanoic acid, compound (7), of formula:

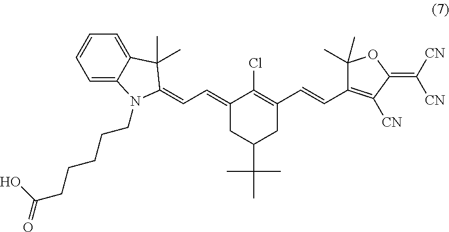 [SMILES: CC1(C)OC(=C(C#N)C#N)C(C#N)=C1/C=C/C1=C(Cl)C(=C/C=C2/N(CCCCCC(=O)O)c3ccccc3C2(C)C)/CC(C(C)(C)C)C1]

6-((E)-2-((E)-2-(5-tert-butyl-3-((E)-2-(4-cyano-5-(dicyanomethylene)-2,2-dimethyl-2,5-dihydrofuran-3-yl)vinyl)-2-((trimethylsilyl)ethynyl)cyclohex-2-enylidene)ethylidene)-3,3-dimethylindolin-1-yl)hexanoic acid, compound (8), of formula:

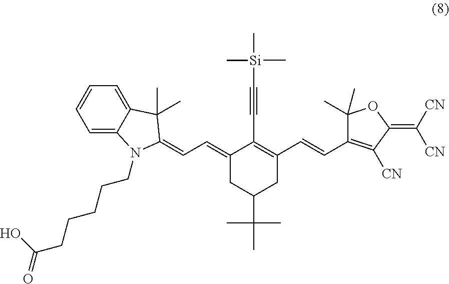 [SMILES: CC1(C)OC(=C(C#N)C#N)C(C#N)=C1/C=C/C1=C(C#C[Si](C)(C)C)C(=C/C=C2/N(CCCCCC(=O)O)c3ccccc3C2(C)C)/CC(C(C)(C)C)C1]

6-((E)-2-((E)-2-(5-tert-butyl-3-((E)-2-(4-cyano-5-(dicyanomethylene)-2,2-dimethyl-2,5-dihydrofuran-3-yl)vinyl)-2-(4-(3-hydroxypropyl)phenoxy)cyclohex-2-enylidene)ethylidene)-3,3-dimethylindolin-1-yl)hexanoic acid, compound (9), of formula:

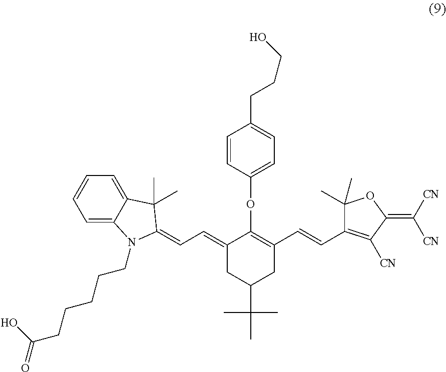 [SMILES: CC1(C)OC(=C(C#N)C#N)C(C#N)=C1/C=C/C1=C(Oc2ccc(CCCO)cc2)C(=C/C=C2/N(CCCCCC(=O)O)c3ccccc3C2(C)C)/CC(C(C)(C)C)C1]

6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)-N-(4-((trimethylsilyl)ethynyl)phenyl)hexanamide, compound (10), of formula:

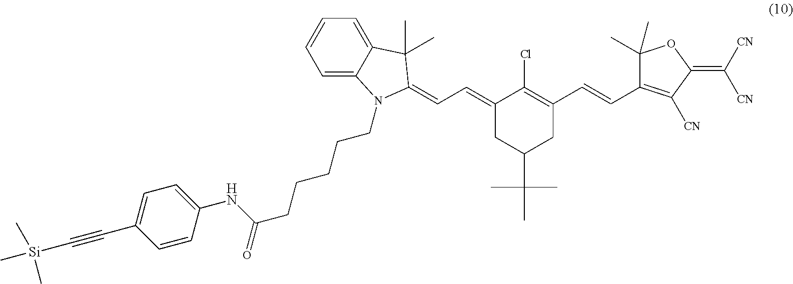 [SMILES: CC1(C)OC(=C(C#N)C#N)C(C#N)=C1/C=C/C1=C(Cl)C(=C/C=C2/N(CCCCCC(=O)Nc3ccc(C#C[Si](C)(C)C)cc3)c3ccccc3C2(C)C)/CC(C(C)(C)C)C1]

6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)-N-(3-hydroxypropyl)hexanamide, compound (11), of formula:

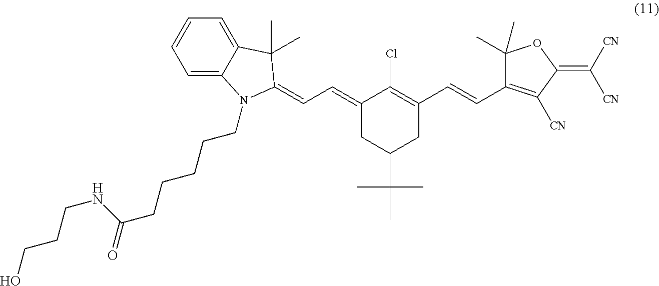 [SMILES: CC1(C)OC(=C(C#N)C#N)C(C#N)=C1/C=C/C1=C(Cl)C(=C/C=C2/N(CCCCCC(=O)NCCCO)c3ccccc3C2(C)C)/CC(C(C)(C)C)C1]

2-(4-((E)-2-((E)-3-((E)-2-(1-(4-bromobenzyl)-3,3-dimethylindolin-2-ylidene)ethylidene)-5-tert-butyl-2-chlorocyclohex-1-enyl)vinyl)-3-cyano-5,5-dimethylfuran-2(5H)-ylidene)malononitrile, compound (12), of formula:

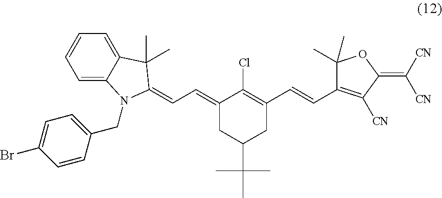 [SMILES: CC1(C)OC(=C(C#N)C#N)C(C#N)=C1/C=C/C1=C(Cl)C(=C/C=C2/N(Cc3ccc(Br)cc3)c3ccccc3C2(C)C)/CC(C(C)(C)C)C1]

2-(4-((E)-2-((E)-3-((E)-2-(1-(4-bromobenzyl)-3,3-dimethylindolin-2-ylidene)ethylidene)-5-tert-butyl-2-(4-(3-hydroxypropyl)phenoxy)cyclohex-1-enyl)vinyl)-3-cyano-5,5-dimethylfuran-2(5H)-ylidene)malononitrile, compound (13), of formula:

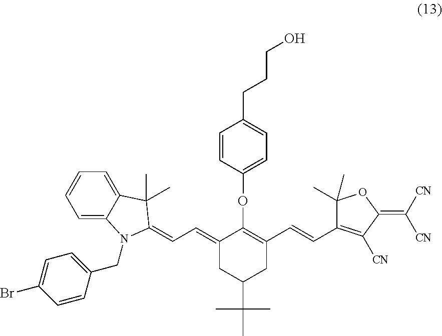 [SMILES: CC1(C)OC(=C(C#N)C#N)C(C#N)=C1/C=C/C1=C(Oc2ccc(CCCO)cc2)C(=C/C=C2/N(Cc3ccc(Br)cc3)c3ccccc3C2(C)C)/CC(C(C)(C)C)C1]

2-(4-((E)-2-((E)-5-tert-butyl-3-((E)-2-(3,3-dimethyl-1-(4-((trimethylsilyl)ethynyl)benzyl)indolin-2-ylidene)ethylidene)-2-(4-(3-hydroxypropyl)phenoxy)cyclohex-1-enyl)vinyl)-3-cyano-5,5-dimethylfuran-2(5M-ylidene)malononitrile, compound (14), of formula:

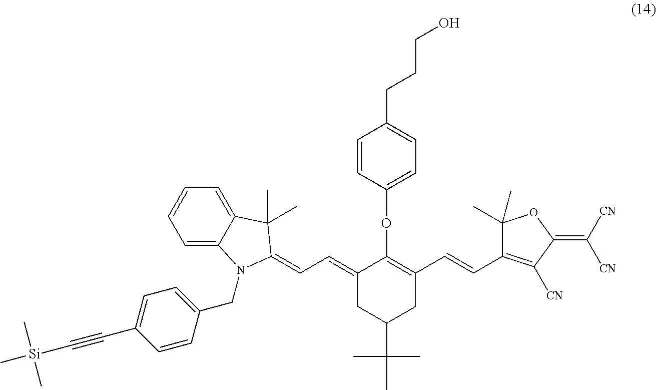 [SMILES: CC1(C)OC(=C(C#N)C#N)C(C#N)=C1/C=C/C1=C(Oc2ccc(CCCO)cc2)C(=C/C=C2/N(Cc3ccc(C#C[Si](C)(C)C)cc3)c3ccccc3C2(C)C)/CC(C(C)(C)C)C1]

2-(4-((E)-2-((E)-3-((E)-2-(1-benzyl-3,3-dimethylindolin-2-ylidene)ethylidene)-5-tert-butyl-2-(4-(3-azidopropyl)phenoxy)cyclohex-1-enyl)vinyl)-3-cyano-5,5-dimethylfuran-2(5H)-ylidene)malononitrile, compound (21), of formula:

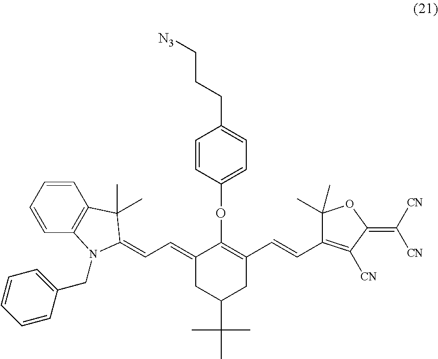 [SMILES: CC1(C)OC(=C(C#N)C#N)C(C#N)=C1/C=C/C1=C(Oc2ccc(CCCN=[N+]=[N-])cc2)C(=C/C=C2/N(Cc3ccccc3)c3ccccc3C2(C)C)/CC(C(C)(C)C)C1]

N-(3-azidopropyl)-6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)hexanamide, compound (22), of formula:

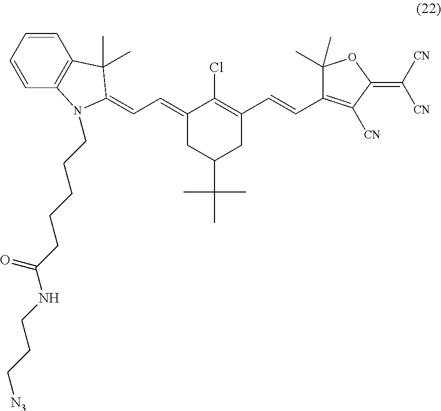 [SMILES: CC1(C)OC(=C(C#N)C#N)C(C#N)=C1/C=C/C1=C(Cl)C(=C/C=C2/N(CCCCCC(=O)NCCCN=[N+]=[N-])c3ccccc3C2(C)C)/CC(C(C)(C)C)C1]

N-(4-azidophenyl)-6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)hexanamide, compound (23), of formula:

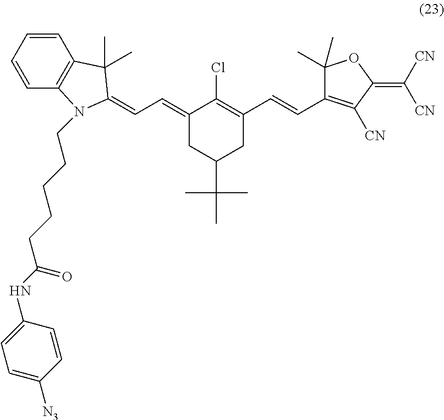 [SMILES: CC1(C)OC(=C(C#N)C#N)C(C#N)=C1/C=C/C1=C(Cl)C(=C/C=C2/N(CCCCCC(=O)Nc3ccc(N=[N+]=[N-])cc3)c3ccccc3C2(C)C)/CC(C(C)(C)C)C1]

6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)-N-(4-(3-hydroxyethyl)phenyl)hexanamide, compound (24), of formula:

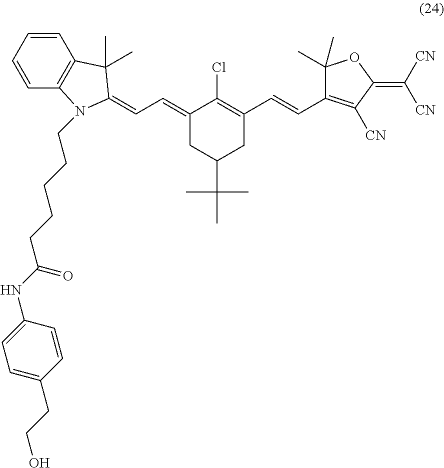 [SMILES: CC1(C)OC(=C(C#N)C#N)C(C#N)=C1/C=C/C1=C(Cl)C(=C/C=C2/N(CCCCCC(=O)Nc3ccc(CCO)cc3)c3ccccc3C2(C)C)/CC(C(C)(C)C)C1]

N-(4-azidophenyl)-6-((E)-2-((E)-2-(5-tert-butyl-3-((E)-2-(4-cyano-5-(dicyanomethylene)-2,2-dimethyl-2,5-dihydrofuran-3-yl)vinyl)-2-(4-(3-hydroxypropyl)phenoxy)cyclohex-2-enylidene)ethylidene)-3,3-dimethylindolin-1-yl)hexanamide, compound (25), of formula:

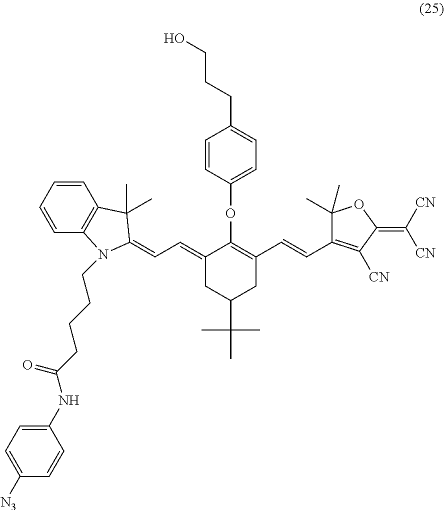 [SMILES: CC1(C)OC(=C(C#N)C#N)C(C#N)=C1/C=C/C1=C(Oc2ccc(CCCO)cc2)C(=C/C=C2/N(CCCCC(=O)Nc3ccc(N=[N+]=[N-])cc3)c3ccccc3C2(C)C)/CC(C(C)(C)C)C1]

N-(4-azidophenyl)-6-((E)-2-((E)-2-(5-tert-butyl-3-((E)-2-(4-cyano-5-(dicyanomethylene)-2,2-dimethyl-2,5-dihydrofuran-3-yl)vinyl)-2-((trimethylsilyl)ethynyl)cyclohex-2-enylidene)ethylidene)-3,3-dimethylindolin-1-yl)hexanamide, compound (26), of formula:

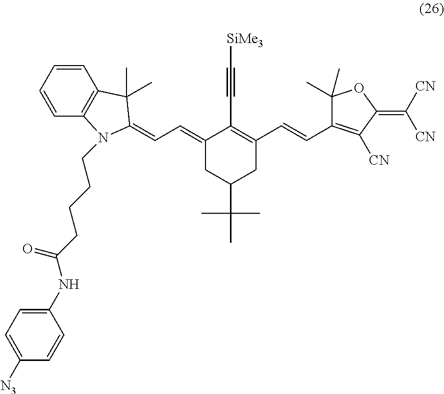 [SMILES: CC1(C)OC(=C(C#N)C#N)C(C#N)=C1/C=C/C1=C(C#C[Si](C)(C)C)C(=C/C=C2/N(CCCCC(=O)Nc3ccc(N=[N+]=[N-])cc3)c3ccccc3C2(C)C)/CC(C(C)(C)C)C1]

6-((E)-2-((E)-2-(5-tert-butyl-3-((E)-2-(4-cyano-5-(dicyanomethylene)-2,2-dimethyl-2,5-dihydrofuran-3-yl)vinyl)-2-(4-(3-hydroxypropyl)phenoxy)cyclohex-2-enylidene)ethylidene)-3,3-dimethylindolin-1-yl)-N-(4-((trimethylsilyl)-ethynyl)phenyl)hexanamide, compound (27), of formula:

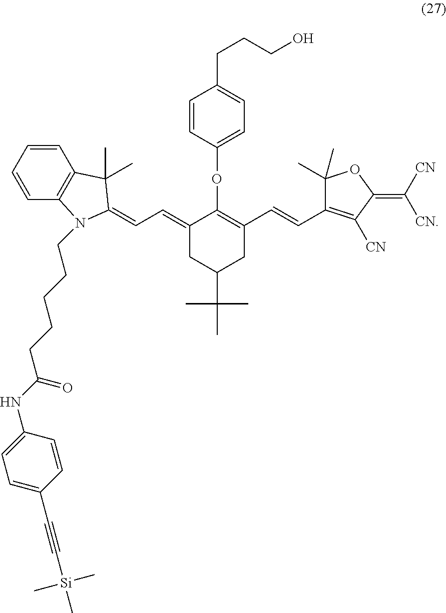 [SMILES: CC1(C)OC(=C(C#N)C#N)C(C#N)=C1/C=C/C1=C(Oc2ccc(CCCO)cc2)C(=C/C=C2/N(CCCCCC(=O)Nc3ccc(C#C[Si](C)(C)C)cc3)c3ccccc3C2(C)C)/CC(C(C)(C)C)C1]

The above chromophores are directionally functionalized or can serve as synthesis intermediate for the preparation of functionalized chromophores which can be integrated by covalent bonding into certain polymers used in electro-optics.

According to an advantageous variant, which may be combined with the previous ones, the chromophores according to the invention comprise a functional group allowing either covalent bonding of the chromophore with a polymer or, once the chromophore has been incorporated into a polymer, crosslinking of the latter, said polymer being in particular chosen from polymers of the polymethacrylate, polyimide, polystyrene, perfluorocyclobutane or polycarbonate type. By way of example, said functional group is chosen from the functional groups carboxylic acid —COOH optionally in protected form, hydroxyl —OH, azide —N₃, amine —NH₂ optionally in protected form, sulfhydryl (—SH), trifluorovinyloxy, ethenyl and the groups —C≡CRf, where Rf is a hydrogen atom or a trialkylsilyl group. According to a specific embodiment, the functional group allowing covalent bonding of a chromophore with a polymer and/or, once the chromophore has been integrated into a polymer, the crosslinking of the latter, is present at the level of the substituent R₃ and/or R₇ of the formula (I). In a particularly preferred manner, the compound of formula (I) according to the invention comprises, at the same time, a functional group allowing covalent bonding of the chromophore with a polymer, and a functional group allowing, once the chromophore has been incorporated into a polymer, crosslinking of the latter, said polymer being chosen in particular from polymers of the polymethacrylate, polyimide, polystyrene, perfluorocyclobutane and polycarbonate type. Said functional groups may be chosen in particular from the functional groups carboxylic acid —COOH optionally in protected form, hydroxyl —OH, azide —N₃, amine —NH₂ optionally in protected form, sulfhydryl (—SH), trifluorovinyloxy, ethenyl and the groups —C≡CRf, where Rf is a hydrogen atom or a trialkylsilyl group. Advantageously, as illustrated in the sole FIGURE, the two functional groups present on the chromophore, one allowing attachment of the chromophore to the polymer (that is to say directly to the polymer already formed, or to a monomer which serves to obtain the desired polymer), the other allowing crosslinking of the polymer, once the chromophore is attached thereto, will be different. Advantageously, one of these functional groups will be located at the level of the substituent R₃ and the other at the level of the substituent R₇. It should be emphasized that the functionalizations at the level of the side chain R₇ do not affect the 2^{nd} order nonlinear optical properties of the chromophores. The substitution at the level of the side chain R₃ by the groups previously defined and in particular the groups —Ophenyl, —Sphenyl and 1H-1,2,3-triazol-4-yl also does not modify the second-order nonlinear optical properties. Compound (2) has the same absorption spectrum and the same μ.β value (29 500×10⁻⁴⁸ esu, measured in chloroform by EFISH at 1907 nm) as the compounds (1a) and (1b) and the absorption spectrum is not modified. The preservation of the second-order nonlinear optical properties can therefore be verified by the measurement of the absorption spectrum which is not modified by the introduction of these substituents.

The description below makes it possible to understand the invention better. To start with, a number of definitions are given as a reminder.

The expression alkyl group is understood to mean a linear or branched saturated hydrocarbon chain. By way of example of an alkyl group comprising from 1 to 12 carbon atoms and in particular from 1 to 7 carbon atoms, there may be mentioned the methyl, ethyl, n-propyl, isopropyl, n-butyl, tert-butyl, sec-butyl, n-pentyl, n-hexyl, n-heptyl and —CH[CH(CH₃)₂]₂ groups.

The expression perfluoroalkyl is understood to mean an alkyl group substituted with one or more fluorine atoms, such as CF₃ for example.

The expression trialkylsilyl is understood to mean a silicon atom substituted with three identical or different alkyl groups of 1 to 6 carbon atoms. By way of example, the trimethylsilyl group may be mentioned. The expression cycloalkyl group denotes a cyclic saturated hydrocarbon chain comprising from 3 to 7 carbon atoms. By way of example of a cycloalkyl group, there may be mentioned the cyclopropyl, cyclobutyl, cyclopentyl, cyclohexyl and cycloheptyl groups.

The expression aryl group is understood to mean a mono-, bi- or polycyclic carbocycle preferably containing from 6 to 12 carbon atoms, comprising at least one aromatic group, for example a phenyl, cinnamyl or naphthyl radical. The phenyl is the particularly preferred aryl group.

Preferably, when the compound of formula (I) contains a substituted benzyl or phenyl group, the latter is substituted with a single substituent at the para position.

The expression functional group —OH, —COOH or —NH₂ in protected form is understood to mean the protecting groups such as those described in Protective Groups in Organic Synthesis, Greene T. W. and Wuts P. G. M., ed John Wiley and Sons, 2006 and in Protecting Groups, Kocienski P. J., 1994, Georg Thieme Verlag.

By way of example of a —NH₂ functional group in protected form, there may be mentioned —NHC(O)OR′₁, with R′₁, which represents an alkyl group of 1 to 12 carbon atoms or a group —(CH₂)ₘ₁R″₁, with R″₁ which represents an aryl (for example phenyl), cycloalkyl or fluorenyl group and ml which is equal to 0, 1, 2 or 3. The functional groups —NHBoc (with Boc=tert-butyloxycarbonyl), —NHFmoc (with =9-fluorenylmethyl-choroformate) and —NHZ (with Z═—C(O)OCH₂Ph) are examples of such functional groups.

By way of example of an —OH functional group in protected form, there may be mentioned the OH functional groups protected with a dihydropyran or a trialkylsilyl or in the form of an ester —OC(O)R′₂ or of an ether —OR′₂, with R′₂ which represents an alkyl group of 1 to 12 carbon atoms or a group —(CH₂)ₘ₂R″₂, with R″₂ which represents an aryl (for example phenyl), cycloalkyl or fluorenyl group and m2 which is equal to 0, 1, 2 or 3.

By way of example of a —COOH functional group in protected form, there may be mentioned the protection as an ester group —C(O)OR′₃, with R′₃ which represents an alkyl group of 1 to 12 carbon atoms or a group —(CH₂)ₘ₂R″₃, with R″₃ which represents an aryl (for example phenyl), cycloalkyl or fluorenyl group and m2 which is equal to 0, 1, 2 or 3.

The subject of the invention is also a method of preparation of chromophores as previously defined, by coupling between a compound of formula (II):

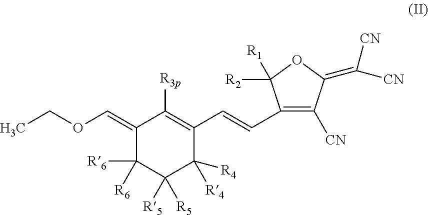

in which R₁, R₂, R₄, R′₄, R₅, R′₅, R₆ and R′₆ are as defined for the compounds of formula (I), with the exception of the compounds (1a) and (1b) and R₃ₚ represents a precursor or protecting group for a group R₃ or a group R₃ as defined for the compounds of formula (I) with the exception of the compounds (1a) and (1b),

and a compound of formula (III):

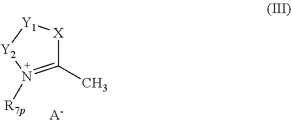

in which X, Y₁ and Y₂ are as defined in claim 1, A- is an anion chosen from Br⁻, I⁻, Cl⁻, methylsulfonate (CH₃SO₃⁻) and para-toluenesulfonate (C₇H₇SO₃⁻), Br being preferred, and R₇ₚ represents a precursor or protecting group for a group R₇ or a group R₇ as defined for the compounds of formula (I) with the exception of the compounds (1a) and (1b).

This reaction is a Knoevenagel coupling. A similar coupling has already been described in the literature, in particular for the synthesis of the compounds (1a) and (1b), known for their 3^{rd} order nonlinear optical properties and developed for optical limitation in the near infrared (Bouit et al. Chem. Mater. 2007, 19 (22), 5325-5335).

For the synthesis of the compounds of formula (Ia), the compound of formula (III) corresponds to the following formula (Ma):

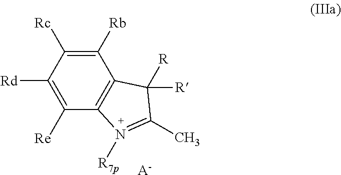

in which R, R′, Rb, Rc, Rd and Re are as defined for the compounds of formula (Ia), with the exception of the compounds (1a) and (1b), A⁻ is an anion chosen from Br⁻, I⁻, Cl⁻, methylsulfonate (CH₃SO₃⁻) and para-toluenesulfonate (C₇H₇SO₃⁻), Br being preferred, and R₇ₚ represents a precursor or protecting group for a group R₇ or a group R₇ as defined for the compounds of formula (Ia) with the exception of the compounds (1a) and (1b).

The coupling of the compounds (II) and (III) or (IIIa) may for example be carried out in a solvent chosen from ethanol, butanol and mixtures thereof, in the presence of pyridine, at the reflux temperature of the solvent used. Such a coupling is preferably carried out under an inert atmosphere, for example under argon.

When the groups R₃ and R₇ are functionalized, the coupling may be carried out between two compounds (II) and (III) bearing groups which are precursors of the desired functional groups. Structural modifications may be made to the simpler chromophores in order to obtain chromophores bearing more complex functionalities, in particular in order to allow their incorporation into various polymer matrices. In particular, it may be advantageous to introduce a site of polymerization such as a hydroxyl or carboxylic acid functional group, as well as a crosslinking site such as a triple bond protected by a silylated group or an azido group, in order to use a Huisgen type 2+2 cycloaddition reaction with the complementary azido or alkyne functional groups of the polymer for the crosslinking (Scarpacci A. et al. Chem. Commun. (Cambridge UK), 2009, 1825-1827). For example, a chlorine atom located at position R₃, or other, may be substituted by various nucleophilic groups (phenolate, thiolate or amines). The substitution of a chlorine atom by a phenolate or a thiophenolate may for example be carried out with potassium carbonate (K₂CO₃) as base in order to deprotonate the phenol, in acetone under argon or with cesium carbonate in anhydrous acetonitrile or alternatively with sodium hydride in THF depending on the phenol or the thiophenol used. The phenolate or thiolate group introduced may comprise a bromine or iodine atom, which can give rise to coupling with trimethylsilylacetylene, under the Sonogashira coupling conditions: in particular in THF, in the presence of PdCl₂(PPh₃)₂, CuI and triethylamine. The phenolate or thiolate group introduced may bear a hydroxyl functional group —OH which may be exchanged for an azido group or esterified with a carboxylic acid.

It is also possible to react a chlorine or bromine atom located at the level of the group R₃ or R₇, or other, with an alkyne functional group, for example present in the trimethylsilylacetylene compound, under the Sonogashira coupling conditions: in particular in THF, in the presence of PdCl₂(PPh₃)₂, CuI and triethylamine. The deprotection of a trimethylsilyl group located at the level of the group R₃ with potassium carbonate and then the reaction with a compound having an azido functional group in the presence of CuSO₄ and sodium ascorbate or of CuI and triethylamine makes it possible to obtain the corresponding compound of formula (I) in which R₃ comprises a 1,2,3-triazole group.

It is also possible to convert a carboxylic acid functional group, for example located at the level of the substituent R₇ in order to give an amide, by the action of an amine in the presence of a coupling agent such as 4-(4,6-dimethoxy-1,3,5-triazin-2-yl)-4-methylmorpholine (DMTMM), at room temperature.

The compounds of formula (II), in which R₃ₚ=Cl, may be obtained in accordance with Scheme 2 given below in the examples, from the corresponding ketone of formula (IV):

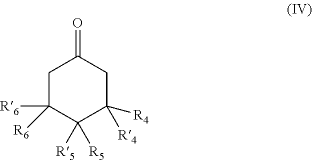

in which R₄, R′₄, R₅, R′₅, R₆ and R′₆ are as defined for the compounds of formula (I).

In particular, the ketones of formula (IV) in which:

R₄═R′₄═R₆═R′₆═H, or R₄═R″₄═R₆═R′₆═—CH₃, and

R₅ and R′₅ are defined as follows:
R₅═R′₅═H; or
R₅═R′₅═—CH₃; or
R₅═H and R′₅ represents a methyl, ethyl, propyl, trifluoromethyl —CF₃, phenyl or tert-butyl group; or
R₅ represents a cyano —CN group and R′₅ represents a phenyl group; or
R₅═H and R′₅ represents a carboxyl —COOH group optionally in protected form; or
R₅ and R′₅ are bonded to each other in order to form the linkage —OCH₂CH₂O—; or
R₅ and R′₅ form with the carbon atom with which they are linked a functional group —C(O)—, correspond to commercial ketones.

The compounds of formula (III), for their part, may be obtained according to Scheme 3 given below in the examples and according to a similar method which will be adapted by a person skilled in the art.

The microscopic nonlinear properties (quadratic hyperpolarizability) of the chromophores according to the invention and the macroscopic properties (electro-optical coefficient) of a polymer material obtained from such chromophores are particularly advantageous. Furthermore, the optical properties of the compounds according to the invention are not affected by the substitutions envisaged, for example such as a phenol, a sulfur atom or a triazole group and these substitutions do not affect the thermal stability of the compounds according to the invention, which is high.

In this context, the subject of the present invention is also the use of a chromophore of formula (I), including a chromophore of formula (Ia) and (1b), for its second-order nonlinear optical properties. In particular, the compound will be used for its high value of the scalar product μ.β where μ represents the dipole moment of the molecule and β the vector value of the quadratic hyperpolarizability, in particular greater than or equal to 30 000×10⁻⁴⁸ esu when it is measured by the EFISH (Electric Field Induced Second Harmonic) technique at 1907 nm in the chloroform. In the context of such a use, the compound of formula (I) may be incorporated into a polymer in order to manufacture an electro-optical modulator.

The chromophores of formula (I) may therefore constitute the active core of an electro-optical modulator, a key device in telecommunication and defense systems. Such an electro-optical modulator may be based on a Mach-Zehnder interferometer built on the basis of nonlinear polymer materials and using the Pockets effect or a resonant architecture of the Fabry-Perot type or Bragg lattice type based on a nonlinear polymer. Only the active guide will consist of a nonlinear polymer obtained incorporating chromophores according to the invention. The chromophores may be introduced into the polymer material in a host-guest system (without covalent attachment). However, according to a particular embodiment, the chromophore will be covalently linked to the polymer. The grafting of the chromophore on to the polymer may be carried out either directly on the polymer, or upstream of its preparation, on the monomers or some of the monomers which, after polymerization, will lead to the desired polymer. The grafting is carried out by reacting the reactive functional group present on the chromophore with a reactive functional group present on the polymer, according to techniques well known to a person skilled in the art. Furthermore, advantageously, the nonlinear polymer will consist of functionalized chromophores according to the invention covalently grafted on to the polymer backbone, oriented under an electric field and crosslinked by the crosslinking functional group carried by the chromophore, as illustrated in the sole FIGURE. This FIGURE schematically represents, first of all, the covalent grafting of a bifunctionalized chromophore (functional groups symbolized A and C in the FIGURE) on to a bifunctionalized polymer matrix (functional groups symbolized B and D in the FIGURE, reacting with the functional groups A and C present on the chromophore, respectively) by post-functionalization or direct polymerization by the chromophore, followed by orientation (poling) under an electric field and finally the crosslinking by the chromophore or the polymer in order to preserve the orientation. The chromophores grafted on polymers may be converted to lattices of hardened materials, thus blocking their electro-optical activity.

A composite incorporating an organic chromophore according to the invention comprises, in a preferred embodiment, a polymer such as a polycarbonate, a polyimide, a hydroxylated polystyrene, a functionalized polymethacrylate or a perfluorocyclobutane. The chromophores according to the invention lead to hardened electro-optical polymers, suitable for electro-optical modulators and other devices such as optical switches. These modulators may be configured so as to operate at high frequencies and in sets for applications in telecommunications and network connections. In addition, they may be used in combinations in series and in parallel in phase controlled radars and in applications for the processing of signals and the technology of detectors.

For example, the chromophores according to the invention, bearing a carboxylic acid —COOH bond, may be introduced into polymer matrices of the hydroxypolystyrene type via an ester linkage. Covalent incorporation into polymer matrices having an azido functional group, of the compounds exhibiting an alkyne functional group via the formation of a triazole is also possible. The covalent incorporation into polymer matrices having an alkyne functional group, of compounds having an azido functional group via the formation of a triazole is also possible. The covalent incorporation into polymer matrices of polyfunctional compounds of formula (I) followed by crosslinking by the formation of a triazole, can also be envisaged.

The examples below, with reference to Schemes 1 to 6, make it possible to illustrate the invention, but are in no way limiting.

The proton NMR (¹H NMR) and carbon 13 (¹³C NMR) spectra were recorded in chloroform-d (CDCl₃) or dimethylsulfoxide-d6 (DMSO-d6) on a Bruker AC200 spectrometer and a Bruker AC500 spectrometer at the frequency of 200 or 500 MHz for the proton and 50 and 125 MHz for carbon 13. The chemical shifts 6 are given using the chemical shift of the solvent as reference. The infrared spectra were recorded on a Mattson 3000 spectrometer using KBr pellets, the absorption spectra were recorded on a Jasco V-670 spectrophotometer, the melting points were measured on a Perkin-Elmer DSC7 calorimeter. The EFISH measurements were carried out on an optical bench according to a method described in the literature [A. Boeglin, A. Fort, L. Mager, C. Combellas, A. Thiébault, V. Rodriguez, Chem. Phys., 2002, 282, 353].

The intermediate 17 is prepared in accordance with Scheme 2 below.

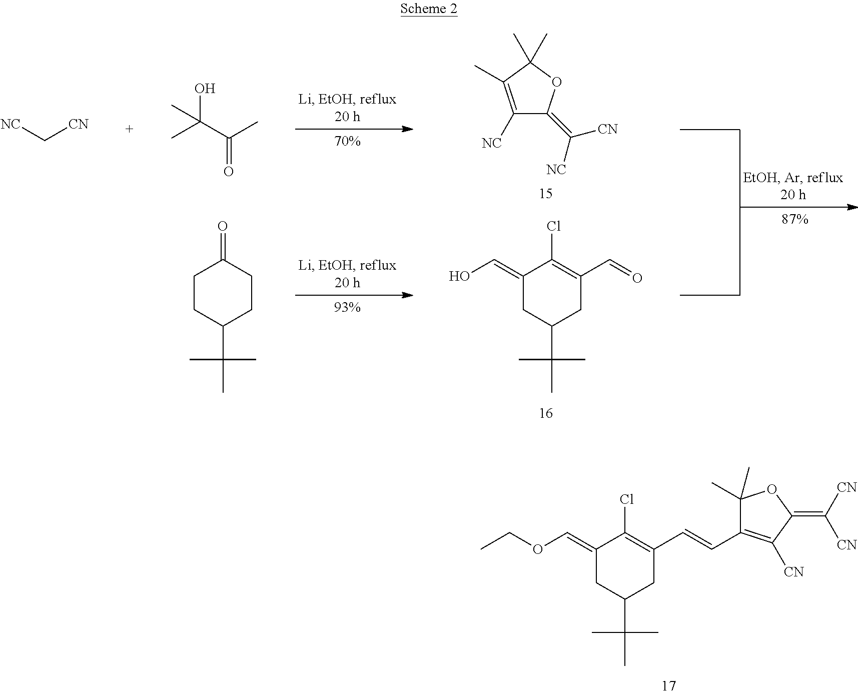

Synthesis of 2-cyanomethylene-3-cyano-4,5,5-trimethyl-2,5-dihydrofuran 15

A 1 L three-necked flask, equipped with a Soxhlet extractor surmounted by a condenser, is flamed under reduced pressure and then purged with argon. An extraction cartridge filled with a 3 Å molecular sieve activated beforehand in an oven at 300° C. is then placed in the Soxhlet extractor. 25 g of 3-hydroxy-3-methylcyclohexanone, 33.15 g of malononitrile (0.5 mol, 2 eq.) and 625 mL of anhydrous ethanol are introduced into the three-necked flask under an argon stream. 30 mg of lithium metal are then added and the mixture is heated under reflux for 20 h. After returning to room temperature, the precipitated solid is filtered, washed with the minimum of cold ethanol and dried. The filtrate is concentrated to half the volume and then placed in a freezer where a second product fraction precipitates, and is combined with the first.

Mass: 25.6 g, 70%, light green solid.

¹H NMR (200 MHz, CDCl₃): δ 1.64 (s, 6H, CH₃), 2.37 (s, 3H, CH₃).

¹³C NMR (50 MHz, CDCl₃): δ 14.0, 24.3, 58.6, 91.2, 99.5, 104.8, 108.8, 110.3, 110.8, 175.0, 182.2.

IR (KBr): 3100, 2230, 1590, 1150, 861 cm⁻¹.

### Synthesis of Bisaldehyde 16

50 mL of anhydrous dimethylformamide (47.4 g, 0.648 mol, 4 eq.) are introduced under an argon atmosphere into a clean dry 250 mL three-necked flask equipped with a dropping funnel, a thermometer and a condenser surmounted by an argon inlet. The solution is cooled to 0° C. with an ice bath, water and salt. 37.8 mL of phosphorus oxychloride (POCl₃, 62.13 g, 0.405 mol, 2.5 eq.) are added dropwise via the dropping funnel, at a rate such that the temperature does not exceed 5° C., and then the mixture is stirred for 15 minutes at 0° C. 25 g of 4-tert-butylcyclohexanone (0.162 ml, 1 eq.) are then gently added to the mixture, still at 0° C. The yellow solution changes to bright red. The cold bath is removed and the mixture is stirred for 10 minutes at room temperature and then heated at 80° C. for 3 hours. After returning to room temperature, the very viscous mixture is gently poured into 500 g of crushed ice with vigorous stirring. A brown gum forms. The flask is placed in an ultrasound bath and the gum is scraped with a spatula for 1 h 30 min to 2 h until a yellow precipitate is obtained. The mixture is then placed for 12 hours in a freezer at −20° C. The solid is filtered on sintered glass, thoroughly washed with water until the pH of the washing solution becomes neutral, and dried for 12 hours under reduced pressure over phosphorus pentoxide (P₂O₅) in a desiccator.

Mass: 34.38 g, 92%, yellow solid. Unstable product to be stored in a freezer at −20° C.

¹H NMR (200 MHz, DMSO-d6): δ 0.88 (s, 9H, t-Bu), 1.22 (m, 1H), 1.75 (dd, 2H, J=12 Hz, J=12 Hz), 1.73 (s, 3H, CH₃), 1.80 (s, 3H, CH₃), 1.98 (m or dd, 1H, J=Hz), 2.59 (d, 1H, J=Hz), 2.76-2.87 (m, 2H), 8.90 (s, 2H).

### Synthesis of the Intermediate 17

A 1 L three-necked flask, equipped with a condenser, is flamed under reduced pressure and then purged with argon. 15.8 g of compound 15 (79.9 mmol, 1 eq.), 20 g of bisaldehyde 16 (87.9 mmol, 1.1 eq.) and 500 mL of anhydrous ethanol are introduced under an argon stream. The solution immediately becomes purple and the mixture forms into a mass.

250 mL of anhydrous ethanol are added. The mixture is heated under reflux for 12 hours. After returning to room temperature, a purple solid is filtered, washed with cold ethanol and dried for 12 hours under reduced pressure over phosphorus pentoxide (P₂O₅) in a desiccator.

Mass: 32.88 g, 87%, red solid.

¹H NMR (200 MHz, CDCl₃): δ 0.97 (s, 9H), 1.36 (t, 3H, J=10 Hz), 1.40 (m, 1H), 1.75 (s, 6H), 1.82 (dd, 1H, 3J=16 Hz, 2J=16 Hz), 2.08 (dd, 1H, 3J=16 Hz, 21=16 Hz), 2.60 (d, 1H, 3J=16 Hz), 2.92 (d, 1H, 3J=16 Hz), 4.11 (q, 2H, J=10 Hz), 6.44 (d, 1H, 31=15 Hz), 7.18 (s, 1H), 8.04 (d, 1H, 31=15 Hz).

¹³C NMR (50 MHz, CDCl₃): δ 15.5, 25.0, 26.9, 27.0, 27.3, 32.3, 42.1, 70.6, 97.2, 97.9, 110.5, 111.4, 112.2, 113.0, 117.0, 128.2, 142.6, 144.4, 153.6, 174.4.

The indolinium salts (18), (19) and (20) are prepared in accordance with Scheme 3 below.

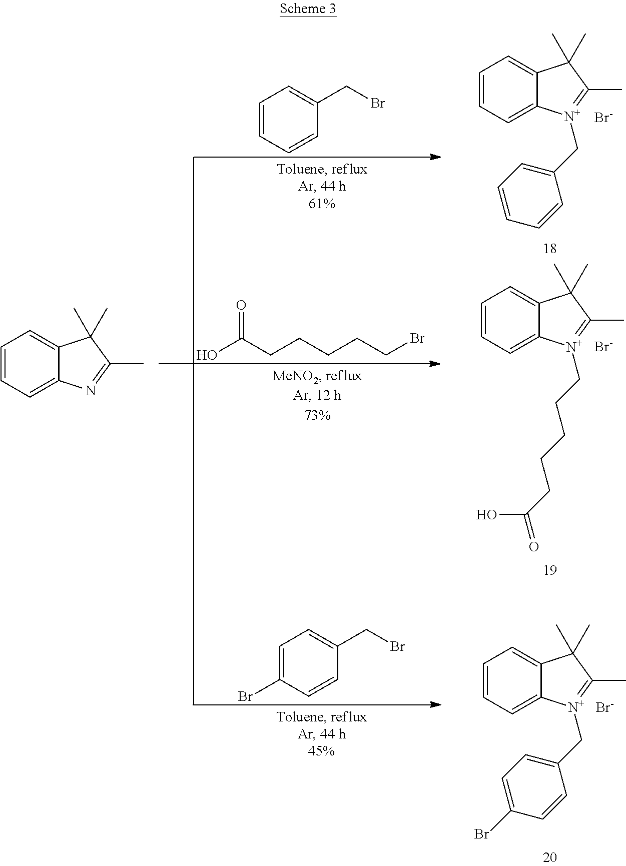

### Synthesis of the Indolinium Salt 18

A 500 mL three-necked flask, equipped with a condenser, is flamed under reduced pressure and then purged with argon. 20 g of freshly distilled 2,3,3-trimethylindolenine (125 mmol, 1 eq.), 30.75 g of benzyl bromide (1.5 eq.) and 250 mL of anhydrous toluene are introduced under an argon stream. The mixture is heated under reflux for 44 h. After returning to room temperature, a pink solid is filtered, washed with acetone and dried for 12 hours under reduced pressure in a desiccator. The filtrate is concentrated by half and then a second fraction is filtered and combined with the first.

Mass: 25.1 g, 61%, pink solid.

¹H NMR (200 MHz, DMSO-d6): δ 1.61 (s, 6H, CH₃), 3.04 (s, 3H, CH₃), 5.90 (m, 2H), 7.38-7.43 (m, 5H), 7.53-7.61 (m, 2H).

¹³C NMR (50 MHz, DMSO-d6): δ 15.3, 22.6, 51.2, 54.9, 116.4, 124.1, 127.8, 129.1, 129.4, 129.7, 129.9, 132.6, 141.5, 142.4, 198.7.

### Synthesis of the Indolinium Salt 19

A 500 mL three-necked flask, equipped with a condenser, is flamed under reduced pressure and then purged with argon. 10 g of freshly distilled 2,3,3-trimethylindolenine (62.89 mmol, 1 eq.), 15.9 g of 6-bromohexanoic acid (1.3 eq.) and 50 mL of nitromethane are introduced under an argon stream. The mixture is heated under reflux for 20 h. After returning to room temperature, ether is added to the mixture until cloudiness appears. The mixture is then subjected to ultrasound treatment until a pale pink precipitate is formed. This solid is filtered, washed with acetone and dried under reduced pressure in a desiccator.

Mass: 16.9 g, 73%, pink solid.

¹H NMR (200 MHz, DMSO-d6): δ 1.61 (s, 6H, CH₃), 3.04 (s, 3H, CH₃), 5.90 (m, 2H), 7.38-7.43 (m, 5H), 7.53-7.61 (m, 2H).

¹³C NMR (50 MHz, DMSO-d6): δ 15.3, 22.6, 51.2, 54.9, 116.4, 124.1, 127.8, 129.1, 129.4, 129.7, 129.9, 132.6, 141.5, 142.4, 198.7.

### Synthesis of the Indolinium Salt 20

A 25 mL three-necked flask, equipped with a condenser, is flamed under reduced pressure and then purged with argon. 1.08 g of freshly distilled 2,3,3-trimethylindolenine (6.78 mmol, 1 eq.), 2.55 g of 4-bromobenzyl bromide (10.20 mmol, 1.5 eq.) and 10 mL of anhydrous toluene are introduced under an argon stream. The mixture is heated under reflux for 12 hours. After returning to room temperature, acetone is added to the reaction mixture until cloudiness appears. The mixture is then subjected to ultrasound treatment until a precipitate forms. The beige solid is filtered, washed with acetone and dried under reduced pressure in a desiccator.

Mass: 1.22 g, 45%, beige solid.

¹H NMR (500 MHz, CDCl₃): δ 1.61 (s, 6H, CH₃), 3.15 (s, 3H, CH₃), 6.09 (2H, s, NCH₂), 7.18 (d, 2H, aromatic CH J=8.4 Hz), 7.52 (d, 2H, aromatic CH J=8.4 Hz), 7.58 (m, 4H).

The chromophores (1a), (7) and (12) are prepared according to Scheme 4 below.

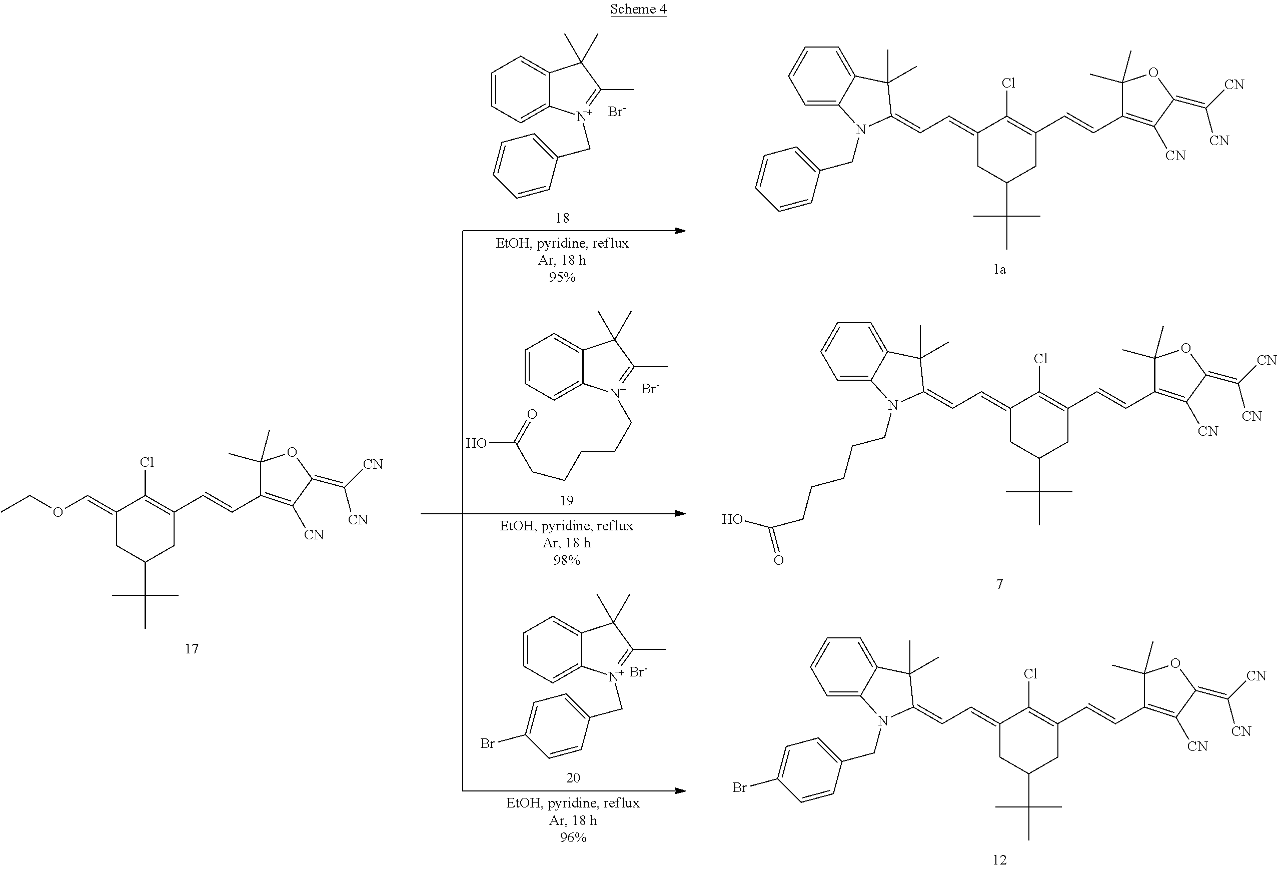

### Synthesis of the Chromophore 1a

A 1 L three-necked flask, equipped with a condenser, is flamed under reduced pressure and then purged with argon. 11.4 g of compound 17 (25.24 mmol, 1 eq.), 10 g of indolinium salt 18 (30.29 mmol, 1.2 eq.), 500 mL of anhydrous ethanol, and then 2.64 mL (2.59 g, 32.81 mmol, 1.3 eq.) of anhydrous pyridine are introduced under an argon stream. The mixture is then heated under reflux overnight. After returning to room temperature, a green solid is filtered, washed with ethanol and dried under reduced pressure in a desiccator.

Mass: 15.34 g, 95%, green solid.

¹H NMR (200 MHz, CDCl3): δ 0.97 (s, 9H), 1.49 (m, 1H), 1.70 (s, 6H), 1.75 (s, 6H), 2.08 (m, 2H), 2.65 (m, 2H), 4.93 (d, 2H, J=10 Hz), 4.99 (d, 1H, J=16 Hz), 5.63 (d, 1H, J=13 Hz), 6.35 (d, 1H, J=16 Hz), 6.88 (d, 1H, J=8 Hz), 7.06, (d, 1H, J=8 Hz), 7.1-7.4 (m, 7H), 7.87 (d, 1H, J=13 Hz), 8.08 (d, 1H, J=16 Hz).

¹³C NMR (50 MHz, CDCl₃): δ 27.5, 27.2, 27.4, 27.6, 28.4, 28.5, 32.3, 42.3, 47.0, 47.4, 94.0, 96.5, 96.9, 108.1, 111.1, 111.6, 112.2, 113.1, 122.2, 122.6, 126.5, 126.7, 128.0, 128.4, 129.2, 135.3, 135.7, 139.6, 143.9, 142.6, 144.4, 146.9, 164.8, 173.2, 176.2.

λₘₐₓ (CH₂Cl₂): 810 nm.

μ.β=31 000×10⁻⁴⁸ esu (EFISH, 1.907 μm in chloroform)

The chromophores (2), (4) (5) and (6) are prepared according to Scheme 5 below.

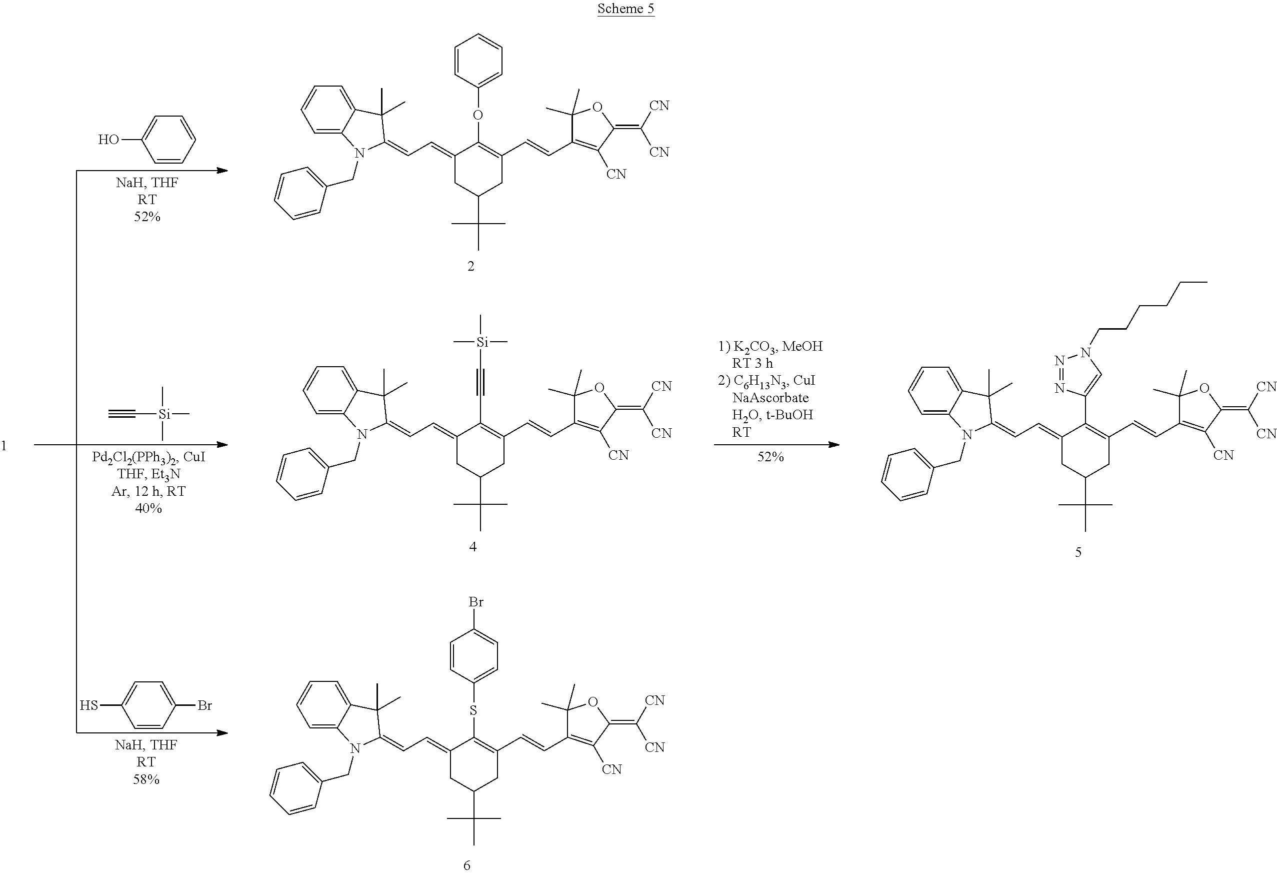

### Synthesis of the Chromophore 2

A 50 mL three-necked flask is flamed under reduced pressure and then purged with argon. 30 mg of phenol (32.5 mmol, 1.2 eq.) in 5 mL of anhydrous THF are slowly added under argon to 20.3 mg of 60% sodium hydride (0.51 mmol, 1.3 eq.) in 3 mL of anhydrous THF at 0° C. The reaction mixture is stirred for 30 minutes, and then it is added to a solution of 250 mg of the chromophore 1a in 10 mL of anhydrous THF. The reaction mixture is stirred for 4 h and then hydrolyzed with a dilute HCl solution. The aqueous phase is extracted with 3×10 mL of dichloromethane. The organic phases are combined, dried over Na₂SO₄, filtered and evaporated under reduced pressure, the product is then purified by column chromatography (SiO₂; CH₂Cl₂) and then dried under reduced pressure in a desiccator.

Mass: 167.25 mg, 52%, green solid.

¹H NMR (500 MHz, CDCl₃): δ 0.98 (s, 9H, CH₃), 1.51-1.62 (m, 13H), 1.90 (m, 1H), 2.15 (m, 1H), 2.65 (m, 1H, CH₂, J=15.4 Hz), 2.75 (m, 2H, CH₂, J=15.2 Hz), 4.96 (m, 2H, NCH₂), 5.60 (d, 1H, J=13.2 Hz), 6.27 (d, 1H, J=15.4 Hz), 6.75 (d, 1H, J=7.9 Hz), 6.80 (m, 2H), 7.08 (d, 1H, J=13.2 Hz), 7.25 (m, 7H), 7.50 (m, 4H).

λₘₐₓ (CH₂Cl₂): 804 nm.

μ.β=29 500×10⁻⁴⁸ esu (EFISH, 1.907 μm in chloroform)

### Synthesis of the Chromophore 4

A 20 mL three-necked flask is flamed under reduced pressure and then purged with argon. 500 mg of the chromophore 1a (0.75 mmol, 1 eq.), 26.32 mg of PdCl₂(PPh₃)₂ (37.49 10⁻³ mmol, 5%), and 14.28 mg of CuI (74.98 10⁻³ mmol, 10%) are introduced under an argon stream and the assembly is then degassed for 15 min. 20 mL of THF are added, followed by 0.22 mL of trimethyl silylacetylene (1.5 mmol, 2 eq.), and finally 5 mL of triethylamine are added. The reaction mixture is stirred for 12 hours at room temperature and under argon, and then hydrolyzed with 20 mL of a saturated NH₄Cl solution. The aqueous phase is extracted with 3×20 mL of dichloromethane. The organic phases are combined, dried over Na₂SO₄, filtered and evaporated under reduced pressure, the product is then purified by column chromatography (SiO₂; CH₂Cl₂/EtOH:10/1) and then dried under reduced pressure in a desiccator.

Mass: 300 mg, 55%, green solid.

¹H NMR (500 MHz, CDCl₃): δ 0.37 (s, 9H, Si(CH₃)₃), 1.05 (s, 9H, CH₃), 1.50 (m, 1H, CH), 1.70-181 (m, 18H), 1.91-2.07 (m, 2H, CH₂), 2.15 (m, 2H, CH₂), 2.73 (d, 1H, CH₂, J=15.8 Hz), 2.83 (d, 1H, CH₂, J=15.8 Hz), 3.82 (m, 2H, NCH₂), 5.74 (d, 1H, J=14.9 Hz), 6.47 (d, 1H, J=15.4 Hz), 6.86 (d, 1H, J=8.3 Hz), 7.07 (t, 1H, J=7.5 Hz, J=7.4 Hz), 7.10 (m, 2H), 8.00 (d, 1H, J=14.9 Hz), 8.06 (d, 1H, J=15.4 Hz).

¹³C NMR (125 MHz, CDCl₃): δ 0.21, 25.5, 25.8, 27.4, 28.5, 32.3, 42.1, 46.9, 47.2, 53.6, 96.2, 96.8, 100.5, 107.5, 108.0, 111.1, 111.4, 112.4, 113.2, 122.1, 122.5, 127.9, 128.5, 131.1, 135.4, 136.4, 137.4, 139.5, 144.0, 146.2, 163.6, 172.9, 176.1.

λₘₐₓ (CH₂Cl₂): 825 nm.

### Synthesis of the Chromophore 5

In a 25 mL flask, a suspension of 100 mg of chromophore 4 (0.14 mmol, 1 eq.), 59 mg of sodium carbonate (K₂CO₃, 0.42 mmol, 3 eq.) in 5 mL of methanol and 2 mL of dichloromethane is stirred at room temperature for 4 h. The mixture is then filtered on silica (elution CH₂Cl₂). 50 mg of a green solid corresponding to the alkyne are obtained. This solid is dissolved in 5 mL of THF and 2 ml of water. 4.9 mg of sodium ascorbate (0.003 mmol, 0.3 eq.), 2.06 mg of copper sulfate (0.008 mmol, 0.1 eq.) and 15.7 mg of 1-azidohexane (0.12 mmol, 1.5 eq.) are added in order. The reaction mixture is stirred for 12 hours, and then hydrolyzed with an NH₄Cl solution, the aqueous phase is extracted with 3×10 mL of dichloromethane, the organic phases are combined, dried over Na₂SO₄, filtered and evaporated under reduced pressure, the product is then purified by column chromatography (SiO₂; CH₂Cl₂) and then dried under vacuum in a desiccator.

Mass: 25 mg, 41% over the two stages, green solid.

¹H NMR (200 MHz, CDCl₃): δ 0.97 (s, 12H), 1.34 (m, 6H), 1.49 (m, 1H), 1.70 (s, 6H), 1.75 (s, 6H), 2.08 (m, 4H), 2.65 (m, 2H), 4.38 (t, 2H, J=10 Hz), 4.93 (d, 2H, J=10 Hz), 4.99 (d, 1H, J=16 Hz), 5.63 (d, 1H, J=13 Hz), 6.35 (d, 1H, J=16 Hz), 6.88 (d, 1H, J=8 Hz), 7.06 (d, 1H, J=8 Hz), 7.1-7.4 (m, 7H), 7.65 (s, 1H), 7.87 (d, 1H, J=13 Hz), 8.08 (d, 1H, J=16 Hz).

λₘₐₓ (CH₂Cl₂): 821 nm

### Synthesis of the Chromophore 6

A 50 mL three-necked flask is flamed under reduced pressure and then purged with argon. 88.5 mg of 4-bromothiophenol (0.46 mmol, 1.2 eq.) in 5 mL of anhydrous THF are slowly added under argon to 20.3 mg of 60% sodium hydride (0.51 mmol, 1.3 eq.) in 3 mL of anhydrous THF at 0° C. The reaction mixture is stirred for 30 minutes, and then it is added to a solution of 250 mg of the chromophore 1a in 10 mL of anhydrous THF. The reaction mixture is stirred for 4 h and then hydrolyzed with a dilute HCl solution. The aqueous phase is extracted with 3×10 mL of dichloromethane. The organic phases are combined, dried over Na₂SO₄, filtered and evaporated under reduced pressure, the product is then purified by column chromatography (SiO₂; CH₂Cl₂) and then dried under reduced pressure in a desiccator.

Mass: 179 mg, 58%, green solid.

¹H NMR (500 MHz, CDCl₃): δ 1.05 (s, 9H, CH₃), 1.50 (m, 1H, CH), 1.69-1.80 (m, 12H), 1.91-2.07 (m, 1H, CH₂), 2.39 (m, 1H, CH₂), 2.67 (d, 1H, CH₂, J=15.8 Hz), 2.80 (d, 1H, CH₂, J=15.8 Hz), 4.82 (m, 2H, NCH₂), 5.74 (d, 1H, J=14.9 Hz), 6.46 (d, 1H, J=13.3 Hz), 6.86 (d, 1H, J=8.3 Hz), 7.08 (t, 1H, J=7.5 Hz, J=7.4 Hz), 7.30 (m, 2H), 7.47 (m, 9H), 8.00 (d, 1H, J=14.9 Hz), 8.06 (d, 1H, J=16.6 Hz).

¹³C NMR (125 MHz, CD₂Cl₂): δ 26.9, 27.2, 27.3, 27.9, 28.2, 28.4, 32.4, 42.4, 47.4, 47.0, 94.0, 96.5, 96.9, 107.9, 111.3, 111.8, 112.2, 112.9, 119.3, 122.1, 122.4, 126.5, 127.7, 127.9, 128.3, 129.2, 130.9, 132.4, 135.4, 136.5, 136.7, 137.2, 139.7, 143.8, 145.9, 146.5, 164.4, 173.4, 176.1.

λₘₐₓ (CH₂Cl₂): 816 nm.

### Synthesis of the Chromophore 7

A 1 L three-necked flask, equipped with a condenser, is flamed under reduced pressure and then purged with argon. 10.3 g of the compound 17 (23.52 mmol, 1 eq.), 10 g of indolinium salt 19 (28.22 mmol, 1.2 eq.), 450 mL of anhydrous ethanol, and then 2.46 mL (2.41 g, 30.57 mmol, 1.3 eq.) of anhydrous pyridine are introduced under an argon stream. The mixture is then heated under reflux overnight. After returning to room temperature, the solution is concentrated until a green solid appears. The latter is filtered, washed with ethanol and dried under reduced pressure in a desiccator.

Mass: 15.4 g, 98%, green solid.

¹H NMR (200 MHz, CDCl₃): δ 1.06 (s, 9H), 1.59 (m, 20H), 2.11 (m, 2H), 2.41 (t, 2H, J=7.1 Hz), 2.76 (d, 1H, J=15 Hz), 2.85 (d, 1H, J=16 Hz), 3.84 (m, 2H, J=16 Hz), 5.70 (d, 1H, J=13.0 Hz), 6.36 (d, 1H, J=15.2 Hz), 6.86 (d, 1H, J=7.8 Hz), 7.09 (t, 1H), 7.28 (m, 2H), 7.96 (d, 1H, J=13.0 Hz), 8.15 (d, 1H, J=15.2 Hz).

λₘₐₓ (CH₂Cl₂): 828 nm.

The synthesis of the functionalized chromophores (8), (10) and (11) is carried out in accordance with Scheme 6 below.

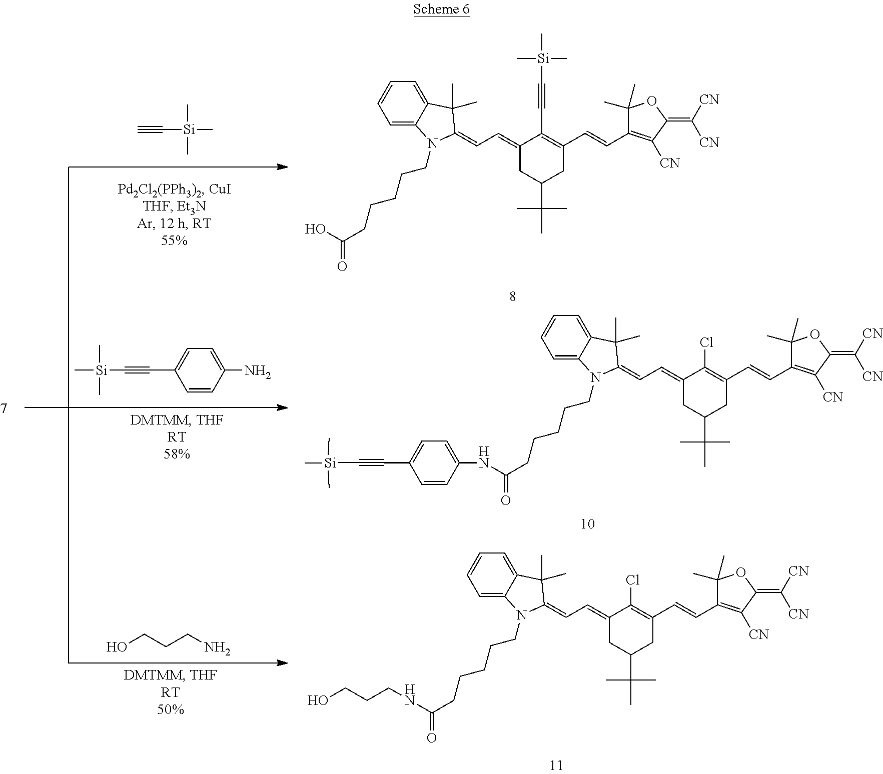

### Synthesis of the Chromophore 8

A 20 mL three-necked flask is flamed under reduced pressure and then purged with argon. 250 mg of compound 7 (0.38 mmol, 1 eq.), 13.68 mg of PdCl₂(PPh₃)₂ (19.10 10⁻³ mmol, 5%), 7.42 mg of CuI (38.00 10⁻³ mmol, 10%) are introduced under an argon stream and the assembly is thus degassed for 15 min. 10 mL of THF are added, followed by 66 μL of trimethyl silylacetylene (0.46 mmol, 1.5 eq.), and then 2 mL of triethylamine are added. The reaction mixture is stirred for 12 hours at room temperature and under argon, and then hydrolyzed with 15 mL of a saturated NH₄Cl solution. The aqueous phase is extracted with 3×15 mL of dichloromethane and the organic phases are combined, dried over Na₂SO₄, filtered and evaporated under reduced pressure, the product is then purified by column chromatography (SiO₂; CH₂Cl₂) and then dried under reduced pressure in a desiccator.

Mass: 110 mg, 40%, green solid.

¹H NMR (500 MHz, CDCl₃): δ 0.36 (s, 9H, Si(CH₃)₃), 0.98 (s, 9H, CH₃), 1.34 (m, 1H, CH), 1.73 (s, 6H, CH₃), 1.78 (s, 6H, CH₃), 1.98 (m, 2H, CH₂), 2.60 (d, 1H, CH₂, J=15.4 Hz), 2.68 (d, 1H, CH₂, J=15.2 Hz), 4.91 (m, 2H, NCH₂), 5.68 (d, 1H, J=13.2 Hz), 6.47 (d, 1H, J=15.4 Hz), 6.90 (d, 1H, J=7.9 Hz), 7.08 (t, 1H, J=7.5 Hz, J=7.4 Hz), 7.10 (m, 7H), 7.89 (d, 1H, J=13.1 Hz), 8.04 (d, 1H, J=15.4 Hz).

¹³C NMR (125 MHz, CDCl₃): δ −0.26, 24.6, 25.5, 25.9, 26.3, 26.8, 27.4, 27.4, 28.4, 32.3, 33.8, 43.2, 42.8, 47.5, 60.4, 95.6, 96.1, 100.6, 107.5, 108.0, 110.7, 111.6, 112.5, 113.4, 122.1, 122.0, 122.3, 128.3, 130.6, 135.9, 137.2, 137.6, 139.7, 143.7, 146.4, 164.3, 172.8, 173.5, 176.4.

λₘₐₓ (CH₂Cl₂): 844 nm.

### Chromophore 10

A 50 mL three-necked flask, 500 mg of chromophore 7 (0.75 mmol, 1 eq.) and 156.5 mg of 4-trimethylsilylethynylaniline (0.82 mmol, 1.1 eq.) in 15 ml of anhydrous THF are stirred for 10 min, and then 229.7 mg of 4-(4,6-dimethoxy-1,3,5-triazin-2-yl)-4-methylmorpholine chloride (DMTMM) are added, the reaction mixture is thus stirred for 24 h, and then hydrolyzed with 15 mL of H₂O. The aqueous phase is extracted with 3×10 mL of dichloromethane. The organic phases are combined, dried over Na₂SO₄; filtered and evaporated under reduced pressure, the product is then purified by column chromatography (SiO₂; CH₂Cl₂/EtOH: 9/1, v/v) and then dried under reduced pressure in a desiccator.

Mass: 350 mg, 56%, green solid.

¹H NMR (500 MHz, CDCl₃): δ 0.25 (s, 9H, Si(CH₃)₃), 1.05 (s, 9H, CH₃), 1.50 (m, 1H, CH), 1.53-1.67 (m, 18H), 1.91-2.07 (m, 2H, CH₂), 2.15 (m, 2H, CH₂), 2.75 (d, 1H, CH₂, J=15.8 Hz), 2.87 (d, 1H, CH₂, J=15.8 Hz), 3.82 (m, 2H, NCH₂), 5.74 (d, 1H, J=14.9 Hz), 6.47 (d, 1H, J=15.4 Hz), 6.86 (d, 1H, J=8.3 Hz), 7.07 (t, 1H, J=7.5 Hz, J=7.4 Hz), 7.10 (m, 2H), 7.47 (m, 4H), 7.97 (d, 1H, J=13.2 Hz), 8.06 (d, 1H, J=12.6 Hz).

¹³C NMR (125 MHz, CD₂Cl₂): δ 0.028, 25.4, 26.9, 27.1, 27.2, 27.5, 27.7, 28.0, 28.4, 28.5, 32.6, 37.6, 42.9, 48.2, 53.8, 93.9, 96.5, 96.9, 104.9, 108.9, 110.5, 112.7, 113.2, 113.8, 118.8, 119.4, 122.4, 123.1, 126.6, 128.2, 128.5, 132.9, 137.7, 138.8, 140.4, 143.7, 144.7, 166.6, 170.9, 173.2, 177.1.

λₘₐₓ (CH₂Cl₂): 833 nm.

### Chromophore 11

A 50 mL three-necked flask, 500 mg of chromophore 7 (0.75 mmol, 1 eq.) and 62.83 mg of aminopropanol (0.82 mmol, 1.1 eq.) in 15 ml of anhydrous THF are stirred for 10 min, and then 229.7 mg of 4-(4,6-dimethoxy-1,3,5-triazin-2-yl)-4-methylmorpholine chloride (DMTMM) are added. The reaction mixture is thus stirred for 24 h, and then hydrolyzed with 15 mL of H₂O, and the aqueous phase is extracted with 3×10 mL of dichloromethane. The organic phases are combined, dried over Na₂SO₄, filtered and evaporated under reduced pressure, the product is then purified by column chromatography (SiO₂; CH₂Cl₂/EtOH: 9/1, v/v) and then dried under reduced pressure in a desiccator.

Mass: 330 mg, green solid.

λₘₐₓ (CH₂Cl₂): 830 nm.

### Synthesis of the Chromophore 12

A 250 mL three-necked flask, equipped with a condenser, is flamed under reduced pressure and then purged with argon. 2.11 g of compound 17 (5.45 mmol, 1 eq.), 2.38 g of indolinium salt 20 (6.55 mmol, 1.2 eq.), 145 mL of anhydrous ethanol, and then 0.58 mL (2.22 mmol, 1.3 eq.) of anhydrous pyridine are introduced under an argon stream. The reaction mixture is then heated under reflux for 12 hours. After returning to room temperature, a red solid is filtered, washed with ethanol and dried under reduced pressure in a desiccator.

Mass: 3.30 g, 95%, red solid

¹H NMR (200 MHz, CDCl₃): δ 0.97 (s, 9H, CH₃), 1.47 (m, 1H, CH), 1.70 (s, 6H, CH₃), 1.75 (s, 6H, CH₃), 1.87 (m, 1H, CH₂), 2.08 (m, 1H, CH₂), 2.60 (d, 1H, CH₂, J=14.3 Hz), 2.65 (d, 1H, CH₂, J=15.6 Hz), 4.91 (m, 2H, NCH₂), 5.53 (d, 1H, J=13.1 Hz), 6.36 (d, 1H, J=15.5 Hz), 6.83 (d, 1H, J=7.9 Hz), 7.07 (t, 1H, J=7.5 Hz, J=7.4 Hz), 7.10 (d, 2H, aromatic CH, J=8.2 Hz), 7.24 (m, 2H), 7.47 (d, 2H, aromatic CH, J=8.3), 7.81 (d, 1H, J=13.1 Hz), 8.09 (d, 1H, J=15.5 Hz).

¹³C NMR (125 MHz, CDCl₃): δ 27.1, 27.3, 27.5, 27.6, 28.3, 28.6, 42.4, 46.4, 47.3, 94.0, 96.5, 96.7, 107.8, 111.4, 111.5, 112.2, 113.1, 121.8, 122.3, 122.6, 126.8, 128.2, 128.3, 128.4, 132.4, 134.4, 135.1, 139.7, 143.7, 144.5, 146.7, 164.1, 173.4, 176.1.

λₘₐₓ (CH₂Cl₂): 794 nm.

The synthesis of the bifunctionalized chromophores (13) and (14) is carried out in accordance with Scheme 7 below.

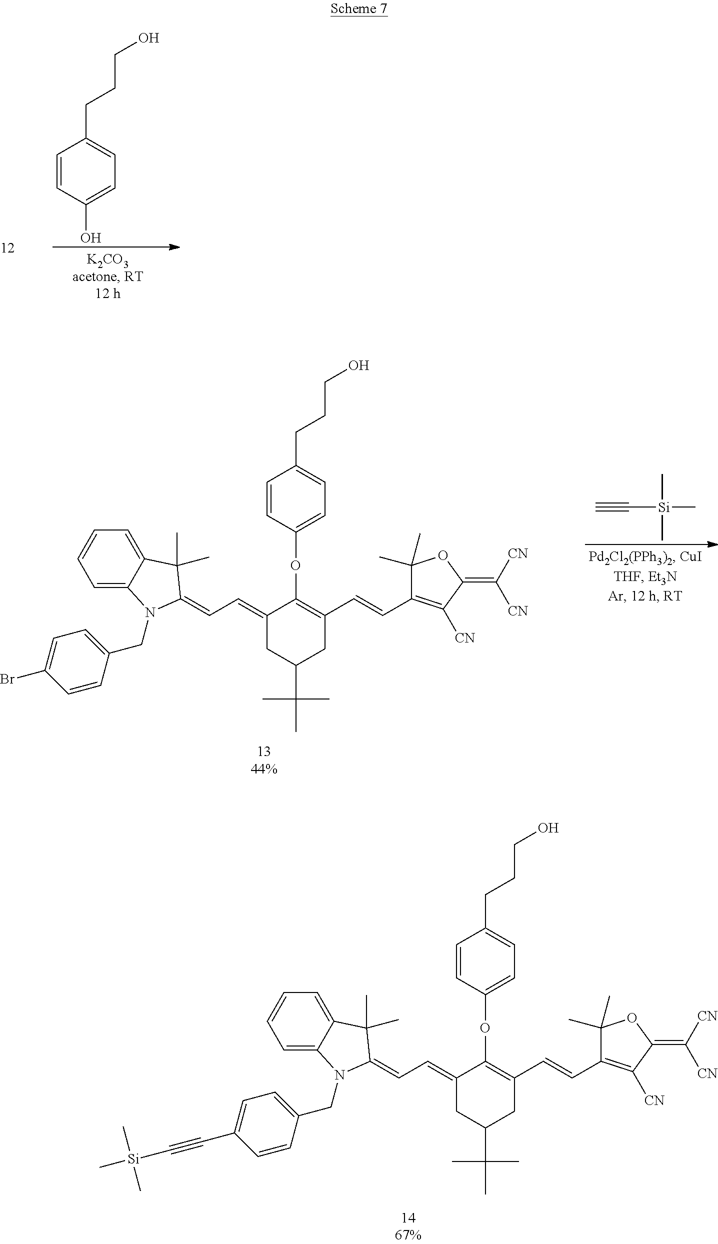

### Synthesis of the Chromophore 13

A 50 mL three-necked flask is flamed under reduced pressure and then purged with argon. 62.4 mg of phenol (87.1 mg, 1.3 eq.) in 3 mL of anhydrous acetone are slowly added under argon to 87.1 mg of potassium carbonate (0.63 mg, 1.4 eq.) in 3 mL of anhydrous acetone at 0° C. The reaction mixture is stirred for 30 minutes, and then it is added to a solution of 250 mg of compound 12 in 9 mL of anhydrous acetone.

The reaction mixture is stirred for 24 h and then neutralized with a dilute HCl solution. The aqueous phase is extracted with 3×10 mL of dichloromethane and the organic phases are combined, dried over Na₂SO₄, filtered and evaporated under reduced pressure, the product is then purified by column chromatography (SiO₂; CH₂Cl₂/MeOH: 98/2, v/v) and then dried under reduced pressure in a desiccator.

Mass: 125 mg, 44%, green solid.

¹H NMR (500 MHz, CDCl₃): δ 1.11 (s, 9H, CH₃), 1.28 (s, 6H, CH₃), 1.42 (s, 6H, CH₃), 1.55 (m, 1H, CH), 1.78 (m, 2H, aliphatic CH₂), 1.95 (m, 1H, ring CH₂) 2.14 (m, 1H, ring CH₂), 2.65 (m, 4H, ring CH₂, aliphatic CH₂), 3.65 (m, 2H, CH₂OH), 4.88 (m, 2H, NCH₂), 5.48 (d, 1H, J=13.3 Hz), 6.25 (d, 1H, J=15.4 Hz), 6.83 (d, 1H, J=7.9 Hz), 6.87 (d, 2H, aromatic CH, J=8.2 Hz), 7.01 (t, 1H, J=7.5 Hz, J=7.4 Hz), 7.08 (d, 2H, J=7.9 Hz), 7.13 (d, 2H, J=8.1 Hz), 7.19 (m, 2H), 7.38 (d, 1H, J=13.2 Hz), 7.45 (d, 2H, aromatic CH, J=8.0), 7.64 (d, 1H, J=15.3 Hz).

λₘₐₓ (CH₂Cl₂): 792 nm.

### Synthesis of the Chromophore 14

A 20 mL three-necked flask is flamed under reduced pressure and then purged with argon. 100 mg of compound 13 (0.12 mmol, 1 eq.), 4.3 mg of PdCl₂(PPh₃)₂ (6.10 10⁻³ mmol, 5%), 2.31 mg of CuI (12.20 10⁻³ mmol, 10%) are introduced under an argon stream and the assembly is degassed by bubbling argon for 15 min. 5 mL of THF are added, followed by 25 μL of trimethyl silylacetylene (0.18 mmol, 1.5 eq.), and finally 2 mL of triethylamine are added. The reaction mixture is stirred for 12 hours at room temperature and under argon, and then hydrolyzed with 10 mL of a saturated NH₄Cl solution. The aqueous phase is extracted with 3×10 mL of dichloromethane, the organic phases are combined, dried over Na₂SO₄, filtered and evaporated under reduced pressure, the product is then purified by column chromatography (SiO₂; CH₂Cl₂/MeOH: 10/0.1, v/v) and then dried under reduced pressure in a desiccator.

Mass: 62 mg, 61%, green solid.

¹H NMR (500 MHz, CDCl₃): δ 0.03 (s, 9H, SiMe₃), 1.11 (s, 9H, CH₃), 1.28 (s, 6H, CH₃), 1.42 (s, 6H, CH₃), 1.55 (m, 1H, CH), 1.78 (m, 2H, aliphatic CH₂), 1.95 (m, 1H, ring CH₂) 2.14 (m, 1H, ring CH₂), 2.65 (m, 4H, ring CH₂, aliphatic CH₂), 3.65 (m, 2H, CH₂OH), 4.88 (m, 2H, NCH₂), 5.48 (d, 1H, J=13.3 Hz), 6.25 (d, 1H, J=15.4 Hz), 6.83 (d, 1H, J=7.9 Hz), 6.87 (d, 2H, aromatic CH, J=8.2 Hz), 7.01 (t, 1H, J=7.5 Hz, J=7.4 Hz), 7.08 (d, 2H, J=7.9 Hz), 7.13 (d, 2H, J=8.1 Hz), 7.19 (m, 2H), 7.38 (d, 1H, J=13.2 Hz), 7.45 (d, 2H, aromatic CH, J=8.0), 7.64 (d, 1H, J=15.3 Hz).

λₘₐₓ (CH₂Cl₂): 791 nm.

The synthesis of the monofunctionalized chromophores (22) to (24) is carried out using DMTMM in accordance with Scheme 8 below.

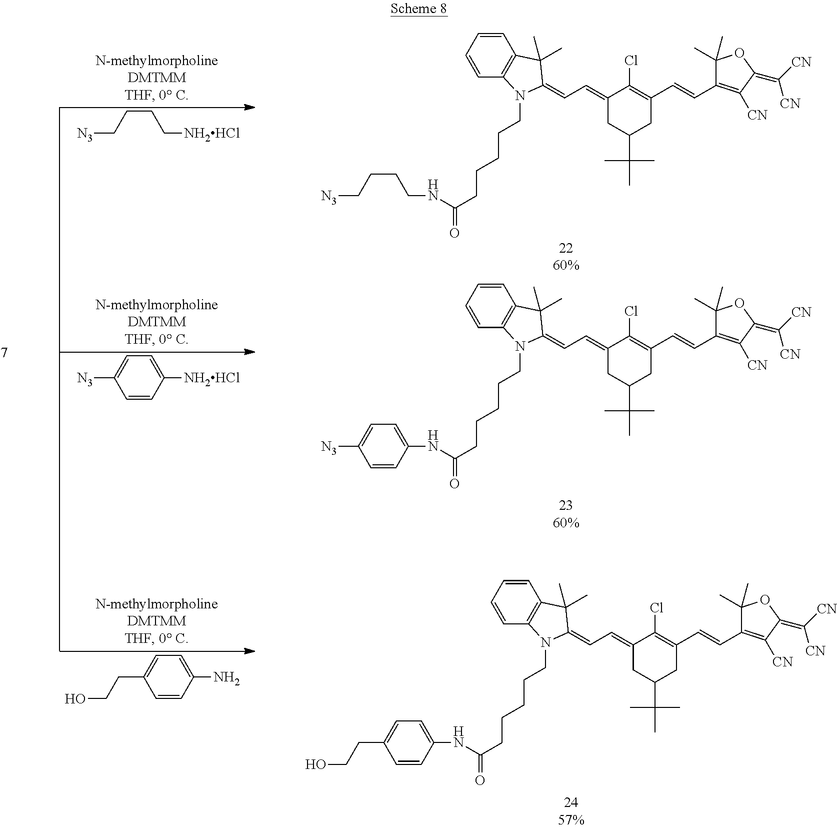

### Synthesis of the Chromophore 22

A solution of 500 mg (0.75 mmol) of compound 7 and 120 mg (0.90 mmol) of 3-azidopropylamine in 15 mL of anhydrous THF is prepared in a flask flamed under vacuum and placed under argon. At room temperature, 250 μL of N-methylmorpholine are added and the solution is stirred for 10 minutes, and then 320 mg (1.15 mmol) of 4-(4,6-dimethoxy-1,3,5-triazin-2-yl)-4-methylmorpholine (DMTMM) are added. The mixture is stirred for 24 h at room temperature. The reaction is stopped by the addition of 5 mL of a dilute HCl solution. The solution is extracted with 3 times 20 mL of dichloromethane. The organic phases are combined, washed successively with 15 mL of water, 15 mL of a saturated NaHCO₃ solution, 15 mL of water, and 15 mL of a saturated NaCl solution. They are then dried over MgSO₄, filtered and evaporated. The crude product is purified by column chromatography (SiO₂, CH₂Cl₂/EtOAc: 98/2, v/v).

Mass: 320 mg (60%), green solid.

¹H NMR (500 MHz, CD₂Cl₂): δ 1.09 (s, 9H), 1.50 (m, 2H, CH₂), 1.60 (m, 3H, CH₂), 1.69 (m, 14H), 1.80 (m, 2H), 2.18 (m, 4H), 2.82 (d, 1H, J=14.3 Hz), 2.93 (d, 1H, J=15 Hz), 3.30 (m, 2H), 3.37 (t, 2H, J=Hz), 3.88 (t, 2H, J=Hz), 5.59 (m, 1H), 5.80 (d, 1H, J=13.3 Hz), 6.33 (d, 1H, J=15.1 Hz), 6.94 (d, 1H, J=8.1 Hz), 7.11 (t, 1H), 7.32 (m, 2H), 8.05 (d, 1H, J=13.3 Hz), 8.30 (d, 1H, J=15.1 Hz).

¹³C NMR (125 MHz, CD₂Cl₂): δ 25.4, 26.5, 26.7, 26.8, 27.0, 27.1, 27.3, 27.6, 27.9, 28.1, 28.9, 32.3, 96.1, 96.6, 108.6, 110.2, 112.2, 112.8, 113.6, 122.1, 122.8, 126.2, 127.6, 128.3, 137.4, 140.1, 143.3, 144.4, 147.5, 166.3, 172.2, 172.8, 176.6.

λₘₐₓ (CH₂Cl₂): 829 nm.

### Synthesis of the Chromophore 23

A solution of 500 mg (0.75 mmol) of compound 7 and 150 mg (0.90 mmol) of 4-azidoaniline in 15 mL of anhydrous THF is prepared in a flask flamed under vacuum and placed under argon. At room temperature, 250 μL of N-methylmorpholine are added and the solution is stirred for 10 minutes, and then 320 mg (1.15 mmol) of 4-(4,6-dimethoxy-1,3,5-triazin-2-yl)-4-methylmorpholine (DMTMM) are added. The mixture is stirred for 24 h at room temperature. The reaction is stopped by the addition of 5 mL of a dilute HCl solution. The solution is extracted with 3 times 20 mL of dichloromethane. The organic phases are combined, washed successively with 15 mL of water, 15 mL of a saturated NaHCO₃ solution, 15 mL of water, and 15 mL of a saturated NaCl solution. They are then dried over MgSO₄, filtered and evaporated. The crude product is purified by column chromatography (SiO₂, CH₂Cl₂/EtOAc: 98/2, v/v).

Mass: 350 mg (60%), green solid.

¹H NMR (500 MHz, CD₂Cl₂): δ 1.05 (s, 9H), 1.50 (m, 3H), 1.66 (s, 6H), 1.74 (s, 6H), 1.80 (m, 4H), 2.14 (m, 2H), 2.36 (t, 2H, J=7.1 Hz), 2.78 (d, 1H, J=17.4 Hz), 2.90 (d, 1H, J=11.7 Hz), 3.88 (t, 2H, J=13.5 Hz, J=9.7 Hz), 5.78 (d, 1H, J=15.7 Hz), 6.27 (d, 1H, J=15.7 Hz), 6.93 (d, 1H, J=7.8 Hz), 6.98 (d, 2H, J=12.6 Hz), 7.09 (t, 1H, J=7.8 Hz, J=7.8 Hz), 7.30 (m, 3H), 7.51 (d, 2H, J=12.6 Hz), 8.02 (d, 1H, J=15.7 Hz), 8.28 (d, 1H, J=15.7 Hz).

¹³C NMR (125 MHz, CD₂Cl₂): δ 25.0, 26.6, 26.7, 26.8, 27.1, 27.2, 27.5, 27.9, 28.1, 32.3, 37.1, 42.5, 43.4, 47.9, 52.1, 91.3, 96.3, 96.5, 108.7, 109.9, 112.5, 113.0, 113.6, 119.8, 121.3, 122.1, 122.8, 126.6, 127.8, 128.7, 135.3, 138.1, 140.4, 143.5, 144.5, 147.9, 166.6, 170.7, 172.8, 176.9.

λₘₐₓ (CH₂Cl₂): 829 nm.

### Synthesis of the Chromophore 24

A solution of 500 mg (0.75 mmol) of compound 7 and 120 mg (0.90 mmol) of 2-(4-aminophenyl)ethanol in 15 mL anhydrous THF is prepared in a flask flamed under vacuum and placed under argon. At room temperature, 250 μL of N-methylmorpholine are added and the solution is stirred for 10 minutes, and then 320 mg (1.15 mmol) of 4-(4,6-dimethoxy-1,3,5-triazin-2-yl)-4-methylmorpholine (DMTMM) are added. The mixture is stirred for 24 h at room temperature. The reaction is stopped by the addition of 5 mL of a dilute HCl solution. The solution is extracted with 3 times 20 mL of dichloromethane. The organic phases are combined, washed successively with 15 mL of water, 15 mL of a saturated NaHCO₃ solution, 15 mL of water, and 15 mL of a saturated NaCl solution. They are then dried over MgSO₄, filtered and evaporated. The crude product is purified by column chromatography (SiO₂, CH₂Cl₂/EtOAc: 9/1, v/v).

Mass: 340 mg (57%), blue solid.

¹H NMR (500 MHz, CD₂Cl₂): δ 1.04 (s, 9H), 1.53 (m, 3H), 1.66 (s, 6H), 1.73 (m, 11H), 1.79 (t, 1H), 1.85 (t, 1H), 2.35 (t, 2H, J=10.0 Hz), 2.82 (m, 3H), 2.90 (m, 1H), 3.78 (t, 2H), 3.88 (m, 2H), 5.83 (m, 1H), 6.24 (m, 1H), 6.94 (d, 1H, J=8.0 Hz), 7.11 (t, 1H, J=7.5 Hz), 7.16 (d, 2H, J=7.9 Hz), 7.30 (t, 2H), 7.45 (d, 2H, J=8.5 Hz), 7.55 (s, 1H), 8.08 (d, 1H, J=15 Hz), 8.29 (m, 1H)

λₘₐₓ (CH₂Cl₂): 829 nm.

The synthesis of the monofunctionalized chromophores (21), (25) and (27) is carried out using cesium carbonate in accordance with Scheme 9 below. The chromophore (13) may also be prepared according to this route using cesium carbonate in anhydrous acetonitrile for the replacement of a chlorine atom by a phenolate.

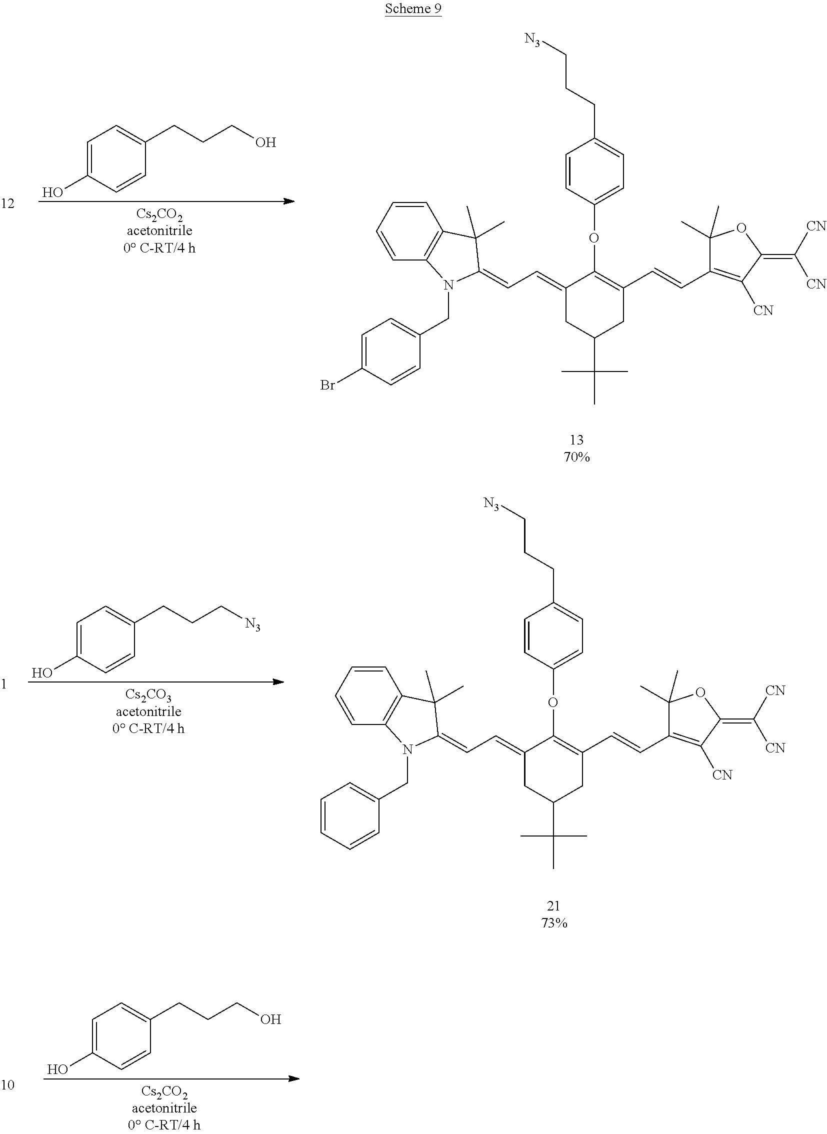 [SMILES: OCCCc1ccc(O)cc1 ; CC1(C)OC(=C(C#N)C#N)C(C#N)=C1/C=C/C1=C(Oc2ccc(CCCN=[N+]=[N-])cc2)C(=C/C=C2/N(Cc3ccc(Br)cc3)c3ccccc3C2(C)C)/CC(C(C)(C)C)C1 ; [N-]=[N+]=NCCCc1ccc(O)cc1 ; CC1(C)OC(=C(C#N)C#N)C(C#N)=C1/C=C/C1=C(Oc2ccc(CCCN=[N+]=[N-])cc2)C(=C/C=C2/N(Cc3ccccc3)c3ccccc3C2(C)C)/CC(C(C)(C)C)C1 ; OCCCc1ccc(O)cc1]

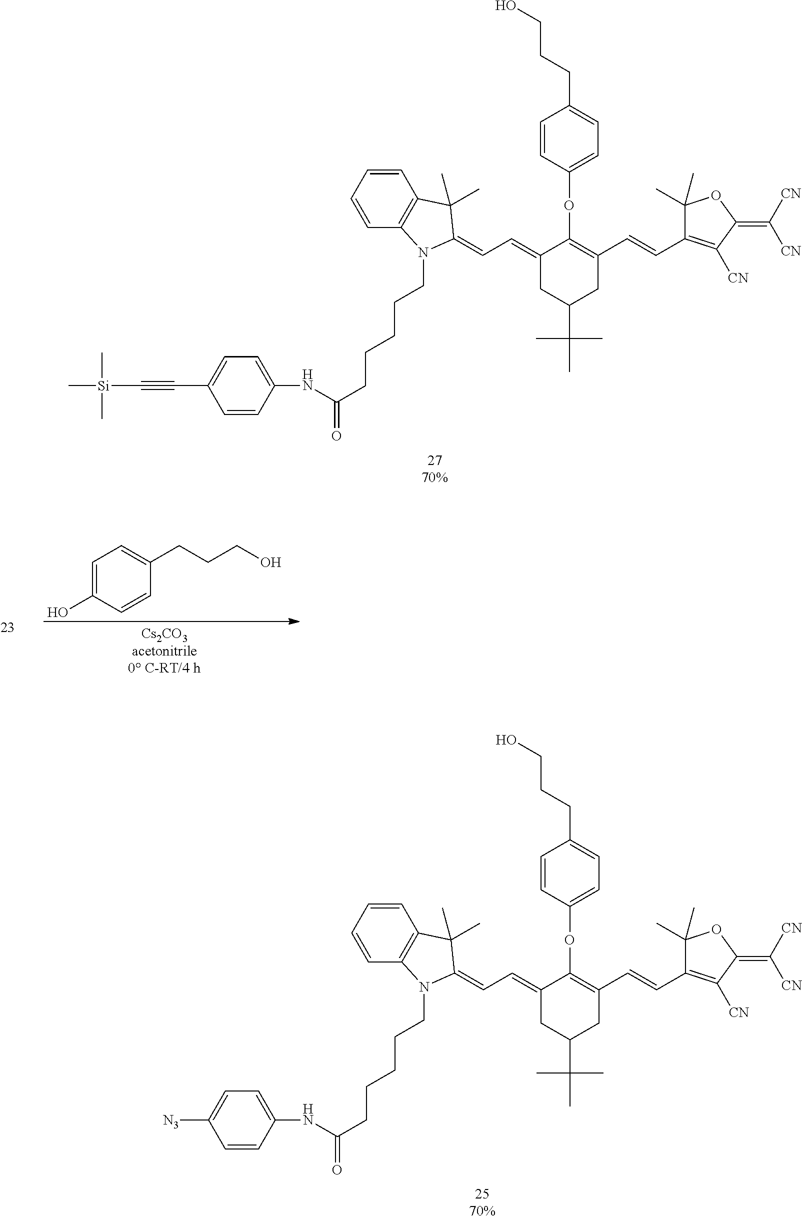 [SMILES: CC1(C)OC(=C(C#N)C#N)C(C#N)=C1/C=C/C1=C(Oc2ccc(CCCO)cc2)C(=C/C=C2/N(CCCCCC(=O)Nc3ccc(C#C[Si](C)(C)C)cc3)c3ccccc3C2(C)C)/CC(C(C)(C)C)C1 ; OCCCc1ccc(O)cc1 ; CC1(C)OC(=C(C#N)C#N)C(C#N)=C1/C=C/C1=C(Oc2ccc(CCCO)cc2)C(=C/C=C2/N(CCCCCC(=O)Nc3ccc(N=[N+]=[N-])cc3)c3ccccc3C2(C)C)/CC(C(C)(C)C)C1]

### Synthesis of the Chromophore 13

A solution of 700 mg (2.16 mmol) of cesium carbonate in 15 mL of anhydrous acetonitrile is prepared and cooled to 0° C. in a two-necked flask flamed under vacuum and purged with argon. A solution of 250 mg (1.66 mmol) of 4-(3-hydroxypropyl)phenol in 5 mL of anhydrous acetonitrile is then added dropwise. After 1 h and after returning to room temperature, a solution of 1.0 g (1.36 mmol) of compound 12 in 10 mL of anhydrous acetonitrile is added and the solution is stirred for 4 h. The solution is neutralized, stopped by the addition of 5 mL of a dilute HCl solution. The solution is extracted with 3 times 20 mL of dichloromethane. The organic phases are combined, dried over MgSO₄, filtered and evaporated. The crude product is purified by column chromatography (SiO₂, CH₂Cl₂/EtOAc: 9/1, v/v).

Mass: 810 mg (70%), green-blue solid.

¹H NMR (500 MHz, CDCl₃): δ 1.11 (s, 9H, CH₃), 1.28 (s, 6H, CH₃), 1.42 (s, 6H, CH₃), 1.55 (m, 1H, CH), 1.78 (m, 2H, CH₂), 1.95 (m, 1H, ring CH₂), 2.14 (m, 1H, ring CH₂), 2.65 (m, 4H, ring CH₂, CH₂), 3.65 (m, 2H, CH₂OH), 4.88 (m, 2H, NCH₂), 5.48 (d, 1H, J=13.3 Hz), 6.25 (d, 1H, J=15.4 Hz), 6.83 (d, 1H, J=7.9 Hz), 6.87 (d, 2H, CHₐᵣ, J=8.2 Hz), 7.01 (t, 1H, J=7.5 Hz, J=7.4 Hz), 7.08 (d, 2H, J=7.9 Hz), 7.13 (d, 2H, J=8.1 Hz), 7.19 (m, 2H), 7.38 (d, 1H, J=13.2 Hz), 7.45 (d, 2H, CHₐᵣ, J=8.0), 7.64 (d, 1H, J=15.3 Hz).

¹³C NMR (125 MHz, CDCl₃): δ 25.1, 25.5, 26.8, 26.8, 27.5, 28.1, 28.2, 31.2, 32.5, 34.4, 42.9, 46.5, 47.1, 62.0, 94.5, 96.0, 96.5, 107.8, 109.9, 111.7, 112.6, 113.5, 114.5, 115.5, 121.8, 122.1, 122.4, 122.5, 122.9, 128.2, 128.3, 129.9, 132.2, 133.1, 134.6, 135.7, 139.5, 142.4, 143.6, 157.8, 161.0, 164.0, 173.3, 176.3.

λₘₐₓ (CH₂Cl₂): 792 nm.

### Synthesis of the Chromophore 21

A solution of 330 mg (1.01 mmol) of cesium carbonate in 8 mL of anhydrous acetonitrile is prepared and cooled to 0° C. in a two-necked flask flamed under vacuum and purged under argon. A solution of 180 mg (1.02 mmol) of 4-(3-azidopropyl)phenol in 2 mL of anhydrous acetonitrile is then added dropwise. After 1 h and after returning to room temperature, a solution of 500 mg (0.77 mmol) of compound 1 in 10 mL of anhydrous acetonitrile is added and the solution is stirred for 8 h. The solution is neutralized, stopped by the addition of 5 mL of a dilute HCl solution. The solution is extracted with 3 times 20 mL of dichloromethane. The organic phases are combined, dried over MgSO₄, filtered and evaporated. The crude product is purified by column chromatography (SiO₂, CH₂Cl₂).

Mass: 441 mg (73%), green solid.

¹H NMR (500 MHz, CDCl₃): δ 1.07 (s, 9H), 1.35 (m, 6H), 1.48 (m, 6H), 1.59 (m, 1H), 1.85 (q, 2H, J=6.9 Hz, J=7.3 Hz, J=14.9 Hz), 2.02 (m, 1H), 2.21 (m, 1H), 2.67 (m, 3H), 2.76 (d, 1H, J=14.1 Hz), 3.25 (t, 2H, J=6.7 Hz, J=13.4 Hz), 4.98 (dd, 2H, J=16.5 Hz, J=36.9 Hz), 5.65 (d, 1H, J=13.3 Hz), 6.25 (d, 1H, J=15.4 Hz), 6.90 (d, 1H, J=7.7 Hz), 6.90 (d, 2H, J=7.8 Hz), 7.09 (t, 1H), 7.16 (d, 2H, J=8.3 Hz), 7.28 (t, 4H), 7.35 (m, 3H), 7.45 (d, 1H, J=13.2 Hz), 7.80 (d, 1H, J=15.2 Hz).

¹³C NMR (125 MHz, CD₂Cl₂): δ 24.2, 25.2, 25.5, 26.5, 27.2, 27.8, 30.7, 31.8, 32.4, 34.2, 42.9, 46.9, 47.2, 50.5, 96.1, 96.6, 108.2, 109.4, 112.1, 112.9, 113.5, 114.6, 115.2, 122.0, 122.4, 122.8, 126.8, 127.8, 128.3, 128.9, 129.2, 129.5, 129.9, 133.7, 134.8, 135.8, 139.7, 142.4, 143.7, 164.4, 173.4, 176.1.

λₘₐₓ (CH₂Cl₂): 803 nm.

### Synthesis of the Chromophore 25

A solution of 163 mg (0.49 mmol) of cesium carbonate in 8 mL of anhydrous acetonitrile is prepared and cooled to 0° C. in a two-necked flask flamed under vacuum and purged with argon. A solution of 59 mg (0.38 mmol) of 4-(3-hydroxypropyl)phenol in 3 mL of anhydrous acetonitrile is then added dropwise. After 1 h and after returning to room temperature, a solution of 250 mg (0.32 mmol) of compound 23 in 10 mL of anhydrous acetonitrile is added and the solution is stirred for 4 h. The solution is neutralized, stopped by the addition of 5 mL of a dilute HCl solution. The solution is extracted with 3 times 20 mL of dichloromethane. The organic phases are combined, dried over MgSO₄, filtered and evaporated. The crude product is purified by column chromatography (SiO₂, CH₂Cl₂/EtOAc: 9/1, v/v).

Mass: 810 mg (70%), green solid.

¹H NMR (500 MHz, CD₂Cl₂): δ 1.12 (s, 9H), 1.30 (m, 6H), 1.47 (s, 8H), 1.68 (m, 1H), 1.80 (m, 6H), 2.20 (m, 2H), 2.36 (m, 2H), 2.56 (m, 2H), 2.80 (d, 1H, J=20.0 Hz), 2.90 (d, 1H, J=20.0 Hz), 3.59 (m, 2H), 3.82 (m, 2H), 5.70 (d, 1H, J=13.0 Hz), 6.22 (d, 1H, J=16.0 Hz), 6.86 (d, 1H, J=7.8 Hz), 6.98 (m, 4H), 7.28 (m, 2H), 7.17 (m, 5H), 7.54 (m, 3H), 7.80 (m, 1H).

¹³C NMR (125 MHz, CD₂Cl₂): δ 24.9, 25.2, 25.5, 26.5, 26.6, 26.7, 27.2, 27.7, 27.8, 31.1, 32.5, 34.5, 37.2, 43.1, 43.2, 47.5, 61.8, 95.3, 96.2, 100.0, 108.4, 112.4, 113.2, 113.8, 114.5, 115.1, 119.4, 121.2, 122.3, 128.2, 129.5, 130.3, 133.1, 134.6, 135.3, 136.2, 140.1, 141.9, 143.2, 158.1, 162.0, 165.9, 170.4, 172.5, 176.6.

λₘₐₓ (CH₂Cl₂): 818 nm.

### Synthesis of the Chromophore 27

A solution of 110 mg (0.34 mmol) of cesium carbonate in 8 mL of anhydrous acetonitrile is prepared and cooled to 0° C. in a two-necked flask flamed under vacuum and purged with argon. A solution of 40 mg (0.26 mmol) of 4-(3-hydroxypropyl)phenol in 1 mL of anhydrous acetonitrile is then added dropwise. After 1 h and after returning to room temperature, a solution of 183 mg (0.22 mmol) of compound 10 in 10 mL of anhydrous acetonitrile is added and the solution is stirred for 8 h. The solution is neutralized, stopped by the addition of 5 mL of a dilute HCl solution. The solution is extracted with 3 times 20 mL of dichloromethane. The organic phases are combined, dried over MgSO₄, filtered and evaporated. The crude product is purified by column chromatography (SiO₂, CH₂Cl₂/EtOAc: 9/1, v/v).

Mass: 810 mg (70%), green-blue solid.

¹H NMR (500 MHz, CD₂Cl₂): δ 0.25 (s, 9H), 1.09 (s, 9H), 1.27 (m, 6H), 1.46 (s, 8H), 1.67 (m, 1H), 1.76 (m, 6H), 2.19 (m, 2H), 2.34 (t, 2H, J=8.5 Hz, J=15.1 Hz), 2.63 (t, 2H, J=6.3 Hz, J=14.6 Hz), 2.78 (d, 1H, J=18.6 Hz), 2.87 (d, 1H, J=18.8 Hz), 3.56 (m, 2H), 3.80 (m, 2H), 5.69 (d, 1H, J=12.5 Hz), 6.19 (d, 1H, J=16.2 Hz), 6.86 (d, 1H, J=10.0 Hz), 6.94 (d, 2H, J=6.3 Hz), 7.03 (t, 1H), 7.17 (m, 4H), 7.24 (t, 1H, J=6.3 Hz, J=12.5 Hz), 7.31 (s, 1H), 7.41 (d, 2H, J=8.7 Hz), 7.48 (d, 2H, J=8.7 Hz), 7.57 (d, 1H, J=13.7 Hz), 7.80 (d, 1H, J=14.9 Hz).

¹³C NMR (125 MHz, CD₂Cl₂): δ −0.26, 24.9, 25.2, 25.5, 26.4, 26.6, 26.7, 27.4, 27.7, 27.9, 31.2, 32.4, 34.6, 37.2, 43.2, 47.5, 61.8, 93.5, 95.5, 96.1, 104.6, 108.4, 112.3, 113.1, 113.7, 114.7, 118.5, 119.1, 121.9, 122.1, 122.3, 122.6, 128.4, 130.1, 132.8, 134.9, 135.8, 138.4, 140.1, 142.4, 143.4, 158.1, 161.8, 166.1, 170.9, 172.6, 176.8.

λₘₐₓ (CH₂Cl₂): 818 nm.

The synthesis of the bifunctionalized chromophore (26) is carried out using DMTMM in accordance with Scheme 10 below.

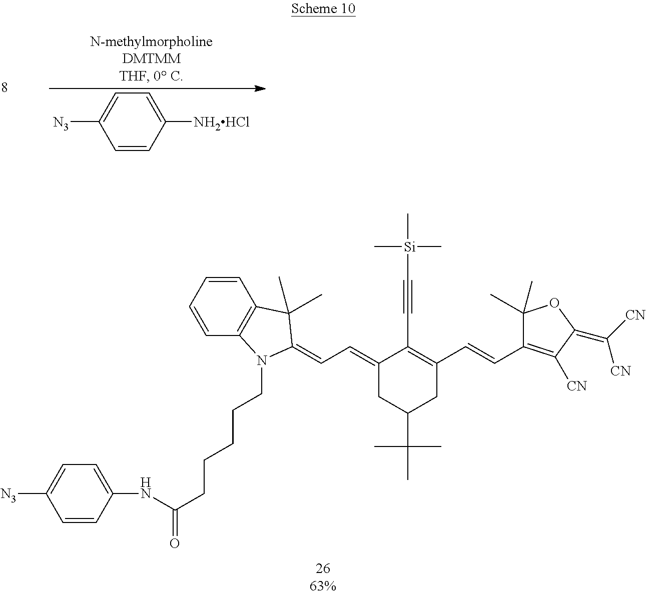

A solution of 275 mg (0.38 mmol) of compound 8 and 78 mg (0.45 mmol) of 4-azidoaniline hydrochloride in 15 mL of anhydrous THF is prepared in a flask flamed under vacuum and placed under argon. At room temperature, 125 μL of N-methylmorpholine are added and the solution is stirred for 10 minutes, then 158 mg (0.57 mmol) of 4-(4,6-dimethoxy-1,3,5-triazin-2-yl)-4-methylmorpholine (DMTMM) are added. The mixture is stirred for 24 h at room temperature. The reaction is stopped by the addition of 5 mL of a dilute HCl solution. The solution is extracted with 3 times 20 mL of dichloromethane. The organic phases are combined, washed successively with 15 mL of water, 15 mL of a saturated NaHCO₃ solution, 15 mL of water, and 15 mL of a saturated NaCl solution. They are then dried over MgSO₄, filtered and evaporated. The crude product is purified by column chromatography (SiO₂, CH₂Cl₂/EtOAc: 9/1, v/v).

Mass: 200 mg (63%), green solid.

¹H NMR (500 MHz, CDCl₃): δ 0.37 (s, 9H), 1.04 (s, 9H), 1.49 (m, 1H), 1.68 (s, 6H), 1.80 (s, 6H), 1.91 (m, 4H), 2.03 (m, 2H), 2.39 (t, 2H, J=7.1 Hz, J=14.3 Hz), 2.73 (d, 1H, J=15.2 Hz), 2.82 (d, 1H, J=14.5 Hz), 3.84 (t, 2H, J=7.2 Hz, J=14.5 Hz), 5.74 (d, 1H, J=13.4 Hz), 6.46 (d, 1H, J=15.3 Hz), 6.86 (d, 1H, J=8.1 Hz), 6.99 (d, 2H, J=8.7 Hz), 7.07 (t, 2H, J=7.3 Hz, J=14.8 Hz), 7.20 (s, 1H), 7.28 (m, 4H), 7.51 (d, 2H, J=8.7 Hz), 7.99 (d, 1H, J=13.2 Hz), 8.07 (d, 1H, J=15.4 Hz)

¹³C NMR (125 MHz, CDCl₃): 0.75, 25.0, 25.97, 26.5, 27.0, 27.3, 27.4, 28.2, 32.8, 37.4, 42.2, 43.2, 47.7, 95.9, 96.2, 101.0, 107.8, 108.4, 110.4, 112.1, 113.7, 119.4, 121.2, 122.0, 128.5, 131.1, 134.8, 135.7, 137.8, 138.1, 139.7, 143.2, 146.2, 165.2, 170.7, 172.7, 176.3.

λₘₐₓ (CH₂Cl₂): 845 nm.

1. A heptamethine hemicyanine type chromophore of formula (Ia): 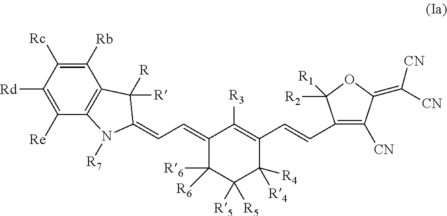
in which:
R₁ and R₂, which are identical or different, represent, each independently of each other, an alkyl group of 1 to 12 carbon atoms, a cycloalkyl group of 3 to 7 carbon atoms, a perfluoroalkyl group of 1 to 12 carbon atoms or a phenyl group, it being possible for said alkyl, perfluoroalkyl, cycloalkyl and phenyl groups to be unsubstituted or substituted with one or more substituents chosen from hydroxyl —OH, carboxylic acid —COOH optionally in protected form, amine —NH₂ optionally in protected form, azide —N₃ and thiol —SH groups,
R₃ represents a hydrogen, chlorine, fluorine, bromine or iodine atom or a group chosen from the groups:
alkyl of 1 to 12 carbon atoms, cycloalkyl of 3 to 7 carbon atoms or perfluoroalkyl of 1 to 12 carbon atoms, it being possible for said alkyl, perfluoroalkyl and cycloalkyl groups to be unsubstituted or substituted with one or more substituents chosen from chlorine, bromine and iodine atoms, and the azide —N₃, hydroxyl —OH, thiol —SH, carboxylic acid —COOH optionally in protected form and —NH₂ optionally in protected form, groups,
—C≡CR₈, in which R₈ represents a hydrogen atom or a trialkylsilyl group or a group chosen from alkyl groups of 1 to 12 carbon atoms, cycloalkyl groups of 3 to 7 carbon atoms or perfluoroalkyl groups of 1 to 12 carbon atoms, it being possible for said alkyl, perfluoroalkyl and cycloalkyl groups to be unsubstituted or substituted with one or more substituents chosen from chlorine, bromine and iodine atoms, and the azide —N₃, hydroxyl —OH, thiol —SH, carboxylic acid —COOH optionally in protected form, and —NH₂ optionally in protected form, groups,
(1H-1,2,3-triazol-4-yl) of formula: 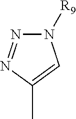
in which R₉ represents a group chosen from alkyl groups of 1 to 12 carbon atoms, cycloalkyl groups of 3 to 7 carbon atoms, perfluoroalkyl groups of 1 to 12 carbon atoms and phenyl groups, it being possible for said alkyl, perfluoroalkyl, cycloalkyl and phenyl groups to be unsubstituted or substituted with one or more substituents chosen from chlorine, bromine and iodine atoms, and the azide —N₃, hydroxyl —OH, thiol —SH, carboxylic acid —COOH optionally in protected form and —NH₂ optionally in protected form, groups, and
—O-phenyl or —S-phenyl, optionally substituted with a chlorine, bromine, iodine or fluorine atom, or with a group chosen from the groups:
azide —N₃,
hydroxyl —OH,
thiol —SH,
carboxylic acid —COOH optionally in protected form,
—NH₂ optionally in protected form,
alkyl of 1 to 6 carbon atoms,
—(CH₂)ₙ—OH, —(CH₂)ₙ—SH, —(CH₂)ₙ—N₃, —(CH₂)ₙ—NH₂ optionally in protected form, —(CH₂)ₙ—COOH optionally in protected form, where n may be equal to 1, 2, 3, 4, 5 or 6,
—C≡CR₁₀, where R₁₀ represents a hydrogen atom or a trialkylsilyl group,
(1H-1,2,3-triazol-4-yl) of formula: 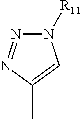
with R₁₁ which represents an alkyl group of 1 to 12 carbon atoms, a cycloalkyl group of 3 to 7 carbon atoms, a perfluoroalkyl group of 1 to 12 carbon atoms or a phenyl group, it being possible for said alkyl, perfluoroalkyl, cycloalkyl and phenyl groups to be unsubstituted or substituted with one or more substituents chosen from the hydroxyl —OH, carboxylic acid —COOH optionally in protected form, amine —NH₂ optionally in protected form, azide —N₃ and thiol —SH groups,
of the dendritic type of general formula: 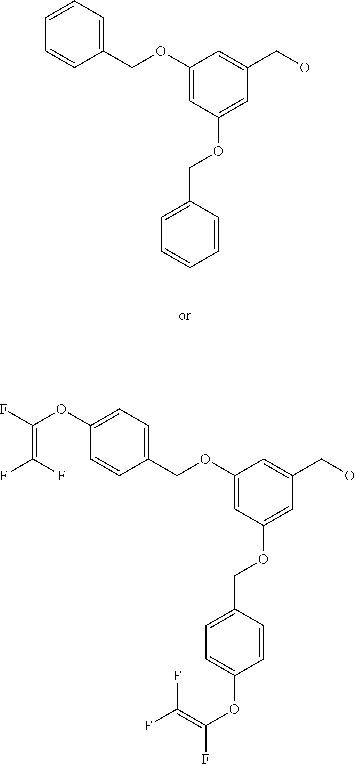
and
—C(O)NHR₁₂, with R₁₂ which represents a —(CH₂)ₚ—OH group with p which may be equal to 1, 2, 3, 4, 5 or 6, or a group chosen from alkyl groups of 1 to 12 carbon atoms, cycloalkyl groups of 3 to 7 carbon atoms, perfluoroalkyl groups of 1 to 12 carbon atoms and phenyl groups, it being possible for said alkyl, cycloalkyl, perfluoroalkyl and phenyl groups to be unsubstituted or substituted with one or more substituents chosen from hydroxyl —OH, carboxylic acid —COOH optionally in protected form, amine —NH₂ optionally in protected form, azide —N₃ and thiol —SH groups,
—O—R₁₃ or —SR₁₃, with R₁₃ which represents an alkyl group of 1 to 12 carbon atoms or a cycloalkyl group of 3 to 7 carbon atoms, it being possible for said alkyl and cycloalkyl groups to be unsubstituted or substituted with one or more substituents chosen from hydroxyl —OH, carboxylic acid —COOH optionally in protected form, amine —NH₂ optionally in protected form, azide —N₃ and thiol —SH groups,
R₄, R′₄, R₅, R′₅, R₆ and R′₆, which are identical or different, represent, each independently of each other:
a hydrogen atom,
a cyano group,
a hydroxyl functional group —OH,
a carboxylic acid functional group —COOH optionally in protected form,
an alkyl group of 1 to 15 carbon atoms, a perfluoroalkyl group of 1 to 15 carbon atoms or a cycloalkyl group of 3 to 7 carbon atoms, it being possible for said alkyl, perfluoroalkyl and cycloalkyl groups to be unsubstituted or substituted with one or more substituents chosen from hydroxyl —OH, carboxylic acid —COOH optionally in protected form, and amine —NH₂ optionally in protected form, groups, or
a phenyl group, optionally substituted with one or more substituents chosen from chlorine atoms and nitrile —CN, carboxylic acid —COOH optionally in protected form, hydroxyl —OH and amine —NH₂ optionally in protected form, groups,
or else R₅ and R′₅ are linked to each other in order to form the linkage —OCH₂CH₂O—; or R₅ and R′₅ form with the carbon atom with which they are linked a —C(O)— functional group, R₄, R′₄, R₆ and R′₆ being as defined above, or else R₄ and R₆ are linked to each other in order to form an alkylene chain comprising 5 or 6 carbon atoms, R′₄, R₅, R′₅ and R′₆ being as defined above, it being understood that in this case, R₅ and R′₅ are not linked to each other in order to form the linkage —OCH₂CH₂O—;
R and R′, which are identical or different, represent, each independently of each other, an alkyl group of 1 to 6 carbon atoms or a cycloalkyl group of 3 to 7 carbon atoms,
Ra represents a hydrogen atom or a methyl group, and Rb, Rc, Rd and Re, which are identical or different, represent, each independently of each other, a hydrogen atom, a hydroxyl —OH group, a carboxylic acid —COOH group optionally in protected form or an amine —NH₂ group optionally in protected form,
R₇ represents a group chosen from the groups:
alkyl of 1 to 12 carbon atoms, cycloalkyl of 3 to 7 carbon atoms and perfluoroalkyl of 1 to 12 carbon atoms, it being possible for said alkyl, perfluoroalkyl and cycloalkyl groups to be unsubstituted or substituted with one or more substituents chosen from chlorine, bromine and iodine atoms, and the azide —N₃, hydroxyl —OH, thiol —SH, carboxylic acid —COOH optionally in protected form and —NH₂ optionally in protected form, groups,
benzyl which is unsubstituted or substituted with one or more substituents chosen from chlorine, bromine and iodine atoms, and the groups:
azide —N₃,
hydroxyl —OH,
carboxylic acid —COOH optionally in protected form,
amine —NH₂ optionally in protected form,
—C≡CR₁₃, with R₁₃, which represents a hydrogen atom, a trialkylsilyl group, an alkyl group of 1 to 12 carbon atoms, a cycloalkyl group of 3 to 7 carbon atoms or a perfluoroalkyl group of 1 to 12 carbon atoms, it being possible for said alkyl, perfluoroalkyl and cycloalkyl groups to be unsubstituted or substituted with one or more substituents chosen from hydroxyl —OH, carboxylic acid —COOH optionally in protected form, amine —NH₂ optionally in protected form, azide —N₃ and thiol —SH, groups,
(1H-1,2,3-triazol-4-yl) of formula: 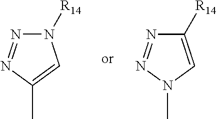
with R₁₄ which represents a group chosen from alkyl groups of 1 to 12 carbon atoms, cycloalkyl groups of 3 to 7 carbon atoms, perfluoroalkyl groups of 1 to 12 carbon atoms and phenyl groups, it being possible for said alkyl, cycloalkyl, perfluoroalkyl and phenyl groups to be unsubstituted or substituted with one or more substituents chosen from hydroxyl —OH, carboxylic acid —COOH optionally in protected form, amine —NH₂ optionally in protected form, azide —N₃ and thiol —SH, groups,
—(CH₂)ₘ—C(O)—R₁₅ with m which is equal to 1, 2, 3, 4, 5 or 6 and R₁₅ which represents —OH or a group —NHR₁₆ with R₁₆ which is chosen from the groups:
phenyl optionally substituted with one or more substituents chosen from chlorine, bromine and iodine atoms, and azide groups —N₃, hydroxyl groups —OH, carboxylic acid groups —COOH optionally in protected form, —NH₂ groups optionally in protected form, and —C≡CR₁₇ groups, with R₁₇ which represents a hydrogen atom, a trialkylsilyl group, an alkyl group of 1 to 12 carbon atoms, a cycloalkyl group of 3 to 7 carbon atoms or a perfluoroalkyl group of 1 to 12 carbon atoms, it being possible for said alkyl, cycloalkyl and perfluoroalkyl groups to be unsubstituted or substituted with one or more substituents chosen from hydroxyl —OH, carboxylic acid —COOH optionally in protected form, amine —NH₂ optionally in protected form, azide —N₃ and thiol —SH, groups,
(1H-1,2,3-triazol-4-yl) of formula: 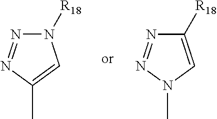
with R₁₈ which represents an alkyl group of 1 to 12 carbon atoms, a cycloalkyl group of 3 to 7 carbon atoms, a perfluoroalkyl group of 1 to 12 carbon atoms, or a phenyl group, it being possible for said alkyl, cycloalkyl, perfluoroalkyl and phenyl groups to be unsubstituted or substituted with one or more substituents chosen from hydroxyl —OH, carboxylic acid —COOH optionally in protected form, amine —NH₂ optionally in protected form, azide —N₃ and thiol —SH, groups,
—(CH₂)_{q}—OH or —(CH₂)_{q}—NH₂, optionally in protected form, with q which may be equal to 1, 2, 3, 4, 5 or 6,
which contains a functional group allowing covalent bonding of the chromophore with a polymer, and/or a functional group allowing, once the chromophore is incorporated into a polymer, crosslinking of the latter, said polymer being in particular chosen from polymers of the polymethacrylate, polyimide, polystyrene, perfluorocyclobutane and polycarbonate type.

2. The chromophore as claimed in claim 1, characterized in that it comprises a functional group allowing covalent bonding of the chromophore with a polymer in particular chosen from polymers of the polymethacrylate, polyimide, polystyrene, perfluorocyclobutane and polycarbonate type.

3. The chromophore as claimed in claim 2, characterized in that it comprises a functional group allowing covalent bonding of the chromophore with a polymer and a functional group allowing, once the chromophore has been incorporated into a polymer, crosslinking of the latter, said polymer being in particular chosen from polymers of the polymethacrylate, polyimide, polystyrene, perfluorocyclobutane and polycarbonate type.

4. The chromophore as claimed in claim 1, characterized in that said polymer is chosen from polymers of the polymethacrylate, polyimide, polystyrene and polycarbonate type.

5. The chromophore as claimed in claim 1, characterized in that said functional group is chosen from the functional groups carboxylic acid —COOH optionally in protected form, hydroxyl —OH, azide —N₃, amine —NH₂ optionally in protected form, sulfhydryl (—SH), trifluorovinyloxy, ethenyl and the groups —C≡CRf, where Rf is a hydrogen atom or a trialkylsilyl group.

6. The chromophore as claimed in claim 1, characterized in that said functional group is chosen from the functional groups carboxylic acid —COOH optionally in protected form, hydroxyl —OH, azide —N₃, amine —NH₂ optionally in protected form and the groups in which Rf is a hydrogen atom or a trialkylsilyl group.

7. The chromophore as claimed in claim 1, characterized in that the said function is present at the level of the substituent R₃ and/or R₇.

8. The chromophore as claimed in claim 1, characterized in that R₁═R₂═CH₃.

9. The chromophore as claimed in claim 1, characterized in that R═R′═CH₃.

10. The chromophore as claimed in claim 1, characterized in that Rb=Rc=Rd=Re═H.

11. The chromophore as claimed in claim 1, characterized in that R₄═R′₄═R₆═R′₆═H or R₄═R′₄═R₆═R′₆═—CH₃.

12. The chromophore as claimed in claim 1, characterized in that R₅ et R′₅ are defined as follows:
R₅═R′₅═H; or
R₅═R′₅=—CH₃; or
R₅═H and R′₅ represents a methyl, ethyl, propyl, trifluoromethyl —CF₃, phenyl or tert-butyl group; or
R₅ represents a cyano —CN group and R′₅ represents a phenyl group; or
R₅═H and R′₅ represents a carboxyl —COOH group optionally in protected form; or
R₅ et R′₅ are linked to each other in order to form the linkage —OCH₂CH₂O—; or
R₅ and R′₅ form with the carbon atom with which they are linked a —C(O)— function.

13. Chromophore chosen among:
2-(4-((E)-2-((E)-3-((E)-2-(1-benzyl-3,3-dimethylindolin-2-ylidene)ethylidene)-5-tert-butyl-2-(phenoxy)cyclohex-1-enyl)vinyl)-3-cyano-5,5-dimethylfuran-2(5H)-ylidene)malononitrile, compound (2), of formula: 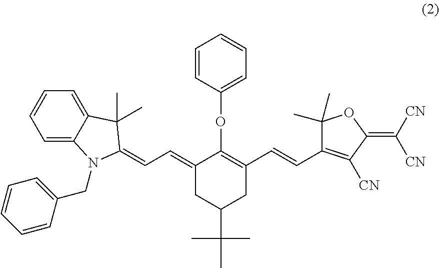 [SMILES: CC1(C)OC(=C(C#N)C#N)C(C#N)=C1/C=C/C1=C(Oc2ccccc2)C(=C/C=C2/N(Cc3ccccc3)c3ccccc3C2(C)C)/CC(C(C)(C)C)C1]
2-(4-((E)-2-((E)-3-((E)-2-(1-benzyl-3,3-dimethylindolin-2-ylidene)ethylidene)-5-tert-butyl-2-(4-(hydroxymethyl)phenoxy)cyclohex-1-enyl)vinyl)-3-cyano-5,5-dimethylfuran-2(5H)-ylidene)malononitrile, compound (3), of formula: 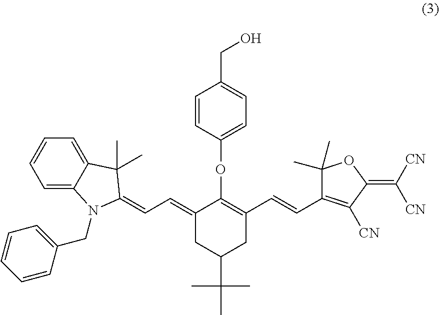 [SMILES: CC1(C)OC(=C(C#N)C#N)C(C#N)=C1/C=C/C1=C(Oc2ccc(CO)cc2)C(=C/C=C2/N(Cc3ccccc3)c3ccccc3C2(C)C)/CC(C(C)(C)C)C1]
2-(4-((E)-2-((E)-3-((E)-2-(1-benzyl-3,3-dimethylindolin-2-ylidene)ethylidene)-5-tert-butyl-2-((trimethylsilyl)ethynyl)cyclohex-1-enyl)vinyl)-3-cyano-5,5-dimethylfuran-2(5H)-ylidene)malononitrile, compound (4), of formula: 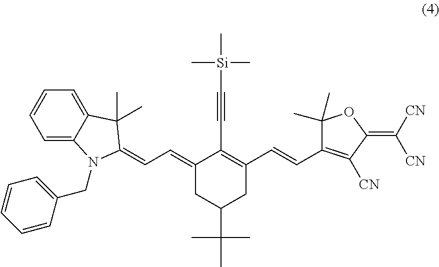 [SMILES: CC1(C)OC(=C(C#N)C#N)C(C#N)=C1/C=C/C1=C(C#C[Si](C)(C)C)C(=C/C=C2/N(Cc3ccccc3)c3ccccc3C2(C)C)/CC(C(C)(C)C)C1]
2-(4-((E)-2-((E)-3-((E)-2-(1-benzyl-3,3-dimethylindolin-2-ylidene)ethylidene)-5-tert-butyl-2-(1-hexyl-1H-1,2,3-triazol-4-yl)cyclohex-1-enyl)vinyl)-3-cyano-5,5-dimethylfuran-2(5H)-ylidene)malononitrile, compound (5), of formula: 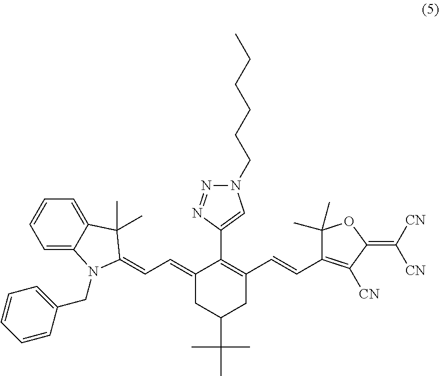 [SMILES: CCCCCCn1cc(C2=C(/C=C/C3=C(C#N)C(=C(C#N)C#N)OC3(C)C)CC(C(C)(C)C)C/C2=C\C=C2\N(Cc3ccccc3)c3ccccc3C2(C)C)nn1]
2-(4-((E)-2-((E)-3-((E)-2-(1-benzyl-3,3-dimethylindolin-2-ylidene)ethylidene)-2-(4-bromophenylthio)-5-tert-butylcyclohex-1-enyl)vinyl)-3-cyano-5,5-dimethylfuran-2(5H)-ylidene)malononitrile, compound (6), of formula: 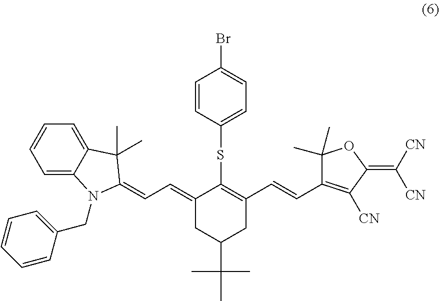 [SMILES: CC1(C)OC(=C(C#N)C#N)C(C#N)=C1/C=C/C1=C(Sc2ccc(Br)cc2)C(=C/C=C2/N(Cc3ccccc3)c3ccccc3C2(C)C)/CC(C(C)(C)C)C1]
acide 6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)hexanoic acid, compound (7), of formula: 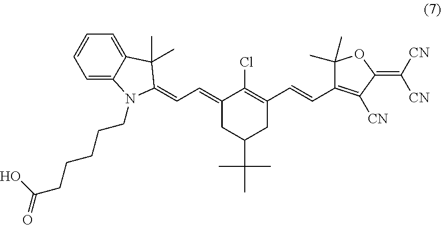 [SMILES: CC1(C)OC(=C(C#N)C#N)C(C#N)=C1/C=C/C1=C(Cl)C(=C/C=C2/N(CCCCCC(=O)O)c3ccccc3C2(C)C)/CC(C(C)(C)C)C1]
acide 6-((E)-2-((E)-2-(5-tert-butyl-3-((E)-2-(4-cyano-5-(dicyanomethylene)-2,2-dimethyl-2,5-dihydrofuran-3-yl)vinyl)-2-((trimethylsilyl)ethynyl)cyclohex-2-enylidene)ethylidene)-3,3-dimethylindolin-1-yl)hexanoic acid, compound (8), of formula: 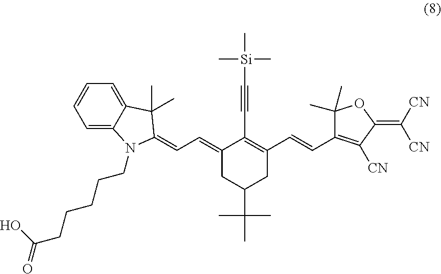 [SMILES: CC1(C)OC(=C(C#N)C#N)C(C#N)=C1/C=C/C1=C(C#C[Si](C)(C)C)C(=C/C=C2/N(CCCCCC(=O)O)c3ccccc3C2(C)C)/CC(C(C)(C)C)C1]
acide 6-((E)-2-((E)-2-(5-tert-butyl-3-((E)-2-(4-cyano-5-(dicyanomethylene)-2,2-dimethyl-2,5-dihydrofuran-3-yl)vinyl)-2-(4-(3-hydroxypropyl)phenoxy)cyclohex-2-enylidene)ethylidene)-3,3-dimethylindolin-1-yl)hexanoic acid, compound (9), of formula: 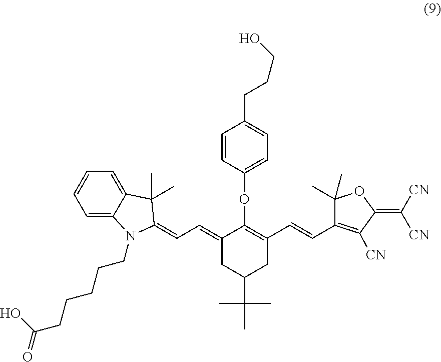 [SMILES: CC1(C)OC(=C(C#N)C#N)C(C#N)=C1/C=C/C1=C(Oc2ccc(CCCO)cc2)C(=C/C=C2/N(CCCCCC(=O)O)c3ccccc3C2(C)C)/CC(C(C)(C)C)C1]
6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)-N-(4-((trimethylsilyl)ethynyl)phenyl)hexanamide, compound (10), of formula: 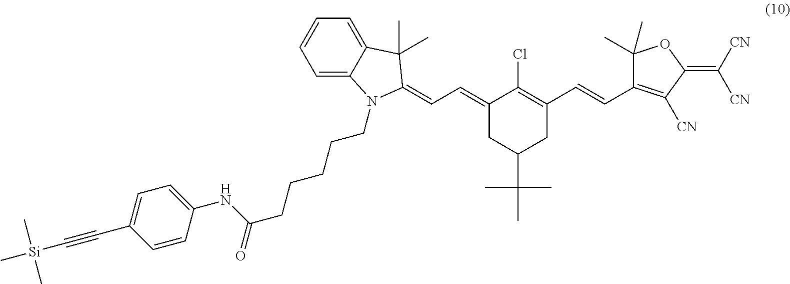 [SMILES: CC1(C)OC(=C(C#N)C#N)C(C#N)=C1/C=C/C1=C(Cl)C(=C/C=C2/N(CCCCCC(=O)Nc3ccc(C#C[Si](C)(C)C)cc3)c3ccccc3C2(C)C)/CC(C(C)(C)C)C1]
6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)-N-(3-hydroxypropyl)hexanamide, compound (11), of formula: 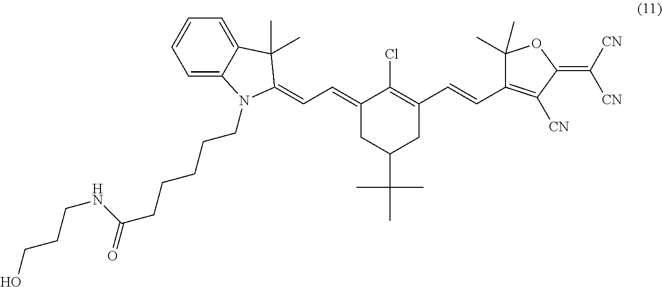 [SMILES: CC1(C)OC(=C(C#N)C#N)C(C#N)=C1/C=C/C1=C(Cl)C(=C/C=C2/N(CCCCCC(=O)NCCCO)c3ccccc3C2(C)C)/CC(C(C)(C)C)C1]
2-(4-((E)-2-((E)-3-((E)-2-(1-(4-bromobenzyl)-3,3-dimethylindolin-2-ylidene)ethylidene)-5-tert-butyl-2-chlorocyclohex-1-enyl)vinyl)-3-cyano-5,5-dimethylfuran-2(5H)-ylidene)malononitrile, compound (12), of formula: 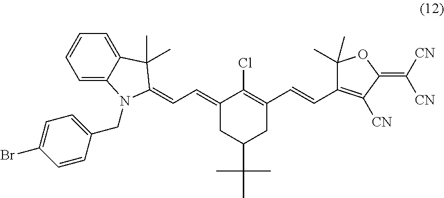 [SMILES: CC1(C)OC(=C(C#N)C#N)C(C#N)=C1/C=C/C1=C(Cl)C(=C/C=C2/N(Cc3ccc(Br)cc3)c3ccccc3C2(C)C)/CC(C(C)(C)C)C1]
2-(4-((E)-2-((E)-3-((E)-2-(1-(4-bromobenzyl)-3,3-dimethylindolin-2-ylidene)ethylidene)-5-tert-butyl-2-(4-(3-hydroxypropyl)phenoxy)cyclohex-1-enyl)vinyl)-3-cyano-5,5-dimethylfuran-2(5H)-ylidene)malononitrile, compound (13), of formula: 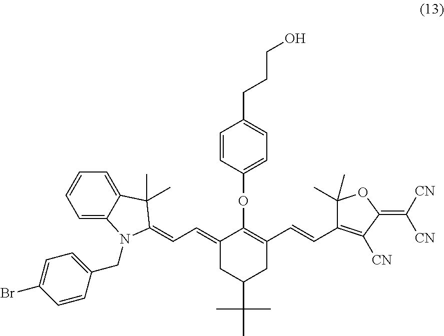 [SMILES: CC1(C)OC(=C(C#N)C#N)C(C#N)=C1/C=C/C1=C(Oc2ccc(CCCO)cc2)C(=C/C=C2/N(Cc3ccc(Br)cc3)c3ccccc3C2(C)C)/CC(C(C)(C)C)C1]
2-(4-((E)-2-((E)-5-tert-butyl-3-((E)-2-(3,3-dimethyl-1-(4-((trimethylsilyl)ethynyl)benzyl)indolin-2-yl idene)ethylidene)-2-(4-(3-hydroxypropyl)phenoxy)cyclohex-1-enyl)vinyl)-3-cyano-5,5-dimethylfuran-2(5H)-ylidene)malononitrile, compound (14), of formula: 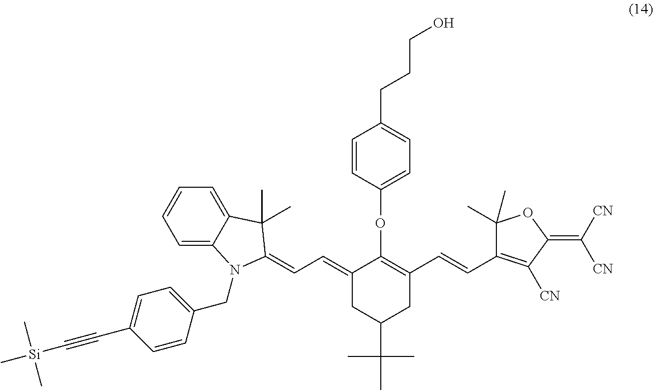 [SMILES: CC1(C)OC(=C(C#N)C#N)C(C#N)=C1/C=C/C1=C(Oc2ccc(CCCO)cc2)C(=C/C=C2/N(Cc3ccc(C#C[Si](C)(C)C)cc3)c3ccccc3C2(C)C)/CC(C(C)(C)C)C1]
2-(4-((E)-2-((E)-3-((E)-2-(1-benzyl-3,3-dimethylindolin-2-ylidene)ethylidene)-5-tert-butyl-2-(4-(3-azidopropyl)phenoxy)cyclohex-1-enyl)vinyl)-3-cyano-5,5-dimethylfuran-2(5H)-yl idene)malononitrile, compound (21), of formula: 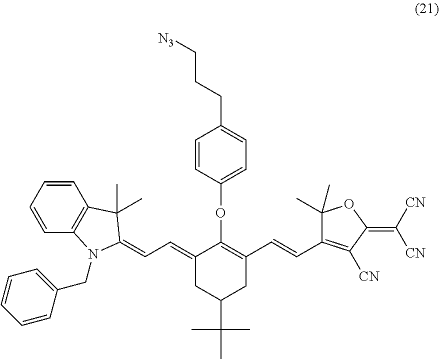 [SMILES: CC1(C)OC(=C(C#N)C#N)C(C#N)=C1/C=C/C1=C(Oc2ccc(CCCN=[N+]=[N-])cc2)C(=C/C=C2/N(Cc3ccccc3)c3ccccc3C2(C)C)/CC(C(C)(C)C)C1]
N-(3-azidopropyl)-6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)hexanamide, compound (22), of formula: 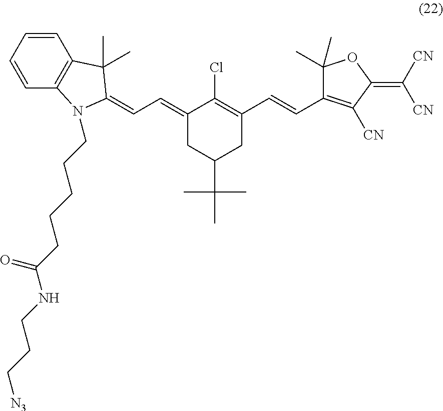 [SMILES: CC1(C)OC(=C(C#N)C#N)C(C#N)=C1/C=C/C1=C(Cl)C(=C/C=C2/N(CCCCCC(=O)NCCCN=[N+]=[N-])c3ccccc3C2(C)C)/CC(C(C)(C)C)C1]
N-(4-azidophenyl)-6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)hexanamide, compound (23), of formula: 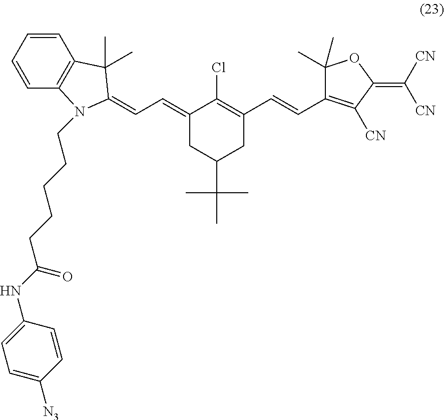 [SMILES: CC1(C)OC(=C(C#N)C#N)C(C#N)=C1/C=C/C1=C(Cl)C(=C/C=C2/N(CCCCCC(=O)Nc3ccc(N=[N+]=[N-])cc3)c3ccccc3C2(C)C)/CC(C(C)(C)C)C1]
6-((E)-2-((E)-2-(5-tert-butyl-2-chloro-3-((E)-2-(4-cyano-5-(dicyanomethylene)-2,2-dimethyl-2,5-dihydrofuran-3-yl)vinyl)cyclohex-2-enylidene)ethylidene)-3,3-dimethylindolin-1-yl)-N-(4-(3-hydroxyethyl)phenyl)hexanamide, compound (24), of formula: 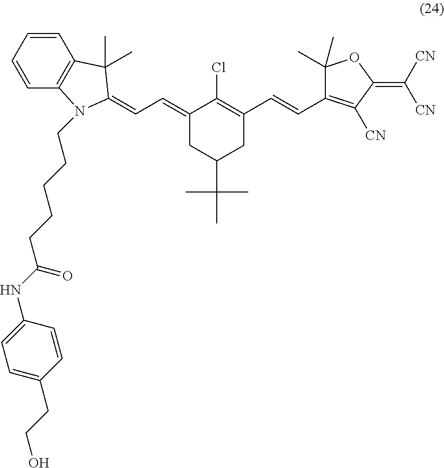 [SMILES: CC1(C)OC(=C(C#N)C#N)C(C#N)=C1/C=C/C1=C(Cl)C(=C/C=C2/N(CCCCCC(=O)Nc3ccc(CCO)cc3)c3ccccc3C2(C)C)/CC(C(C)(C)C)C1]
N-(4-azidophenyl)-6-((E)-2-((E)-2-(5-tert-butyl-3-((E)-2-(4-cyano-5-(dicyanomethylene)-2,2-dimethyl-2,5-dihydrofuran-3-yl)vinyl)-2-(4-(3-hydroxypropyl)phenoxy)cyclohex-2-enylidene)ethylidene)-3,3-dimethylindolin-1-yl)hexanamide, compound (25), of formula: 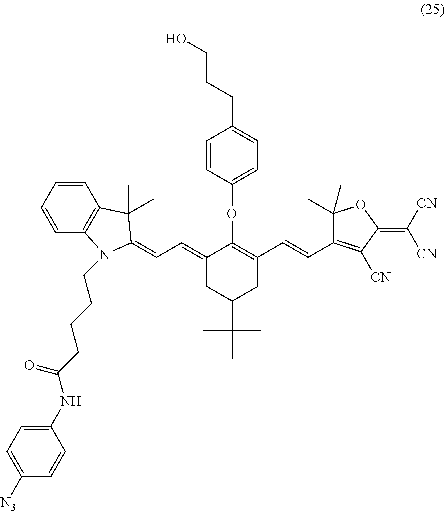 [SMILES: CC1(C)OC(=C(C#N)C#N)C(C#N)=C1/C=C/C1=C(Oc2ccc(CCCO)cc2)C(=C/C=C2/N(CCCCC(=O)Nc3ccc(N=[N+]=[N-])cc3)c3ccccc3C2(C)C)/CC(C(C)(C)C)C1]
N-(4-azidophenyl)-6-((E)-2-((E)-2-(5-tert-butyl-3-((E)-2-(4-cyano-5-(dicyanomethylene)-2,2-dimethyl-2,5-dihydrofuran-3-yl)vinyl)-2-((trimethylsilyl)-ethynyl)cyclohex-2-enylidene)ethylidene)-3,3-dimethylindolin-1-yl)hexanamide, compound (26), of formula: 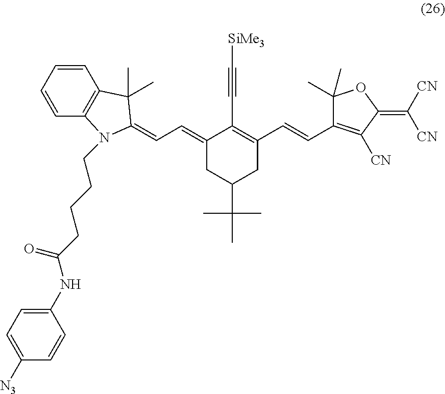 [SMILES: CC1(C)OC(=C(C#N)C#N)C(C#N)=C1/C=C/C1=C(C#C[Si](C)(C)C)C(=C/C=C2/N(CCCCC(=O)Nc3ccc(N=[N+]=[N-])cc3)c3ccccc3C2(C)C)/CC(C(C)(C)C)C1]
6-((E)-2-((E)-2-(5-tert-butyl-3-((E)-2-(4-cyano-5-(dicyanomethylene)-2,2-dimethyl-2,5-dihydrofuran-3-yl)vinyl)-2-(4-(3-hydroxypropyl)phenoxy)cyclohex-2-enylidene)ethylidene)-3,3-dimethylindolin-1-yl)-N-(4-((trimethylsilyl)ethynyl)phenyl)hexanamide, compound (27), of formula:

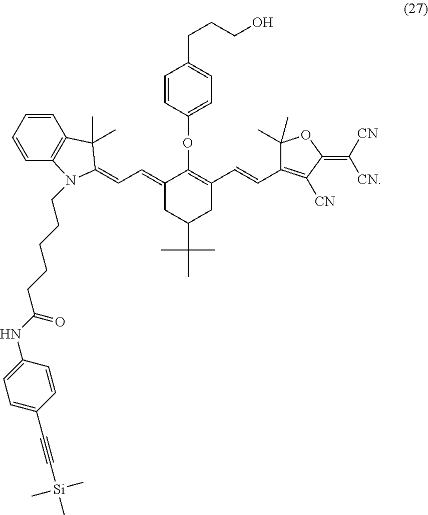 [SMILES: CC1(C)OC(=C(C#N)C#N)C(C#N)=C1/C=C/C1=C(Oc2ccc(CCCO)cc2)C(=C/C=C2/N(CCCCCC(=O)Nc3ccc(C#C[Si](C)(C)C)cc3)c3ccccc3C2(C)C)/CC(C(C)(C)C)C1]

14. A method of preparation of a chromophore as claimed in claim 1, by coupling between a compound of formula (II): 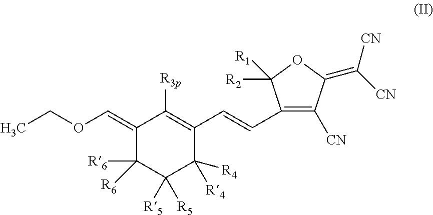
in which R₁, R₂, R₄, R₄, R₅, R′₅, R₆ and R′₆ are as defined in claim 1 and R₃ₚ represents a group R₃ as defined in claim 1 or a precursor or protecting group for such a group R₃, and a compound of the following formula (IIIa): 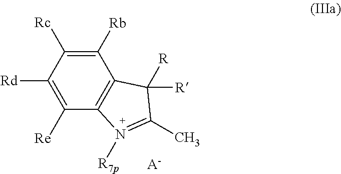
in which R, R′, Rb, Rc, Rd and Re are as defined in claim 1, K is an anion chosen from Br⁻, I⁻, Cl⁻, methylsulfonate (CH₃SO₃⁻) and para-toluenesulfonate (C₇H₇SO₃⁻), Br being preferred, and R₇ₚ represents a group R₇ as defined in claim 1 or a precursor or protecting group for such a group R₇.

15. The method as claimed in claim 14, characterized in that the coupling is carried out in a solvent chosen from ethanol, butanol and mixtures thereof, in the presence of pyridine, at the reflux temperature of the solvent used.

16. The use of a chromophore as claimed in claim 1, for its second-order nonlinear optical properties.

17. The use as claimed in claim 16, characterized in that the chromophore is used for its high value of the scalar product μ.β where μ represents the dipole moment of the molecule and β the vector value of the quadratic hyperpolarizability, in particular greater than or equal to 30 000×10⁻⁴⁸ esu when it is measured by the EFISH (Electric Field Induced Second Harmonic) technique at 1907 nm in the chloroform.

18. The use as claimed in claim 16, characterized in that the chromophore is used for the manufacture of an electro-optical modulator.

19. The use as claimed in claim 16, characterized in that the chromophore is incorporated into a polymer in order to manufacture an electro-optical modulator.

20. The use as claimed in claim 19, characterized in that the polymer is linked to the chromophore by covalent bonding.
